(19) United States
(12) Patent Application Publication
(10) Pub. No.: US 2003/0194373 A1
(43) Pub. Date: Oct. 16, 2003

(54) Angiogenesis targeting molecules

(76) Inventors: Theresa Fauconnier, Toronto (CA); Alfred Pollak, Ljubljana (SI); John Thornback, Stewkley (GB); Dennis Eshima, Keller, TX (US)

Correspondence Address: KRAMER LEVIN NAFTALIS & FRANKEL LLP, INTELLECTUAL PROPERTY DEPARTMENT, 919 THIRD AVENUE, NEW YORK, NY, 10022, US

(21) Appl. No.: 10/420,205

(22) Filed: Apr. 22, 2003

Related U.S. Application Data
(63) Continuation of application No. 09/636,179, filed on Aug. 10, 2000, now abandoned.
Application No. 09/636,179 is a continuation of application No. PCT/CA99/00101, filed on Feb. 11, 1999.
(60) Provisional application No. 60/074,420, filed on Feb. 11, 1998.

(51) Int. Cl. A61K 51/00; C07K 5/06; C07F 5/00

(52) U.S. Cl. 424/1.69; 530/331

(57) ABSTRACT

The present invention relates to compounds that are effective for targeting sites of angiogenesis for diagnostic and therapeutic purposes. The compounds are of the Formula (I) A-(B)n-C, wherein A is a chelator moiety capable of complexing a radionuclide metal or a moiety capable of binding to a halogen; B is a spacer group; C is an angiogenesis targeting molecule; and n is selected from the integers 0 and 1. The invention also relates to a method of imaging sites of angiogenesis and treating patients through the administration of the compounds of the present invention.

This application is a continuation of co-pending application PCT/CA99/00101 filed Feb. 11, 1999 which application claims priority from U.S. provisional patent application S No. 60/074,420 filed Feb. 11, 1998.

### FIELD OF THE INVENTION

The present invention relates to labelled molecules that target proteins or receptors that are upregulated at or on endothelial at sites of angiogenesis. The labelled molecules can be used to detect, stage, and/or treat tumours and metastases. The agents can also be used to detect and treat other angiogenic disorders.

### BACKGROUND OF THE INVENTION

A supply of blood vessels is required for turnouts to grow beyond a few millimeters in diameter and metastasize. The blood supply is accessed by the process called angiogenesis whereby a vascular supply is developed from existing vasculature for the growth, maturation, and maintenance of tissue. It is a complex multistep process which involves the endothelial cells of the lumen of blood vessels. Endothelial cells contain all the information necessary to form tubes, branches, and capillary networks. The process of angiogenesis is also necessary for wound healing and such conditions as diabetic retinopathy, rheumatoid arthritis, and psoriasis.

The sequence of events leading to the formation of new blood vessels has been well documented. One of the first events that occurs is a dissolution of the basement membrane of an existing vessel and the interstitial matrix near a neoplasm by matrix metalloproteinases (MMP-2 and MMP-9). The newly exposed endothelial cells are then induced by growth factors released from the tumour cells to proliferate and migrate towards the growing tumour. The endothelial cells form lumen and then branches and loops of vessels to permit blood flow through the cancerous mass. Basement membrane is then added to the immature vessel.

The quiescence or angiogenic activity of endothelial cells depends on a balance between molecules that stimulate angiogenesis and those that inhibit it. When the balance is switched to promote angiogenic activity, there are many intra- and extracellular proteins and receptors that are overexpressed. Most of the factors responsible for angiogenesis have been discovered in the last decade. Not surprisingly, as promoters and inhibitors of angiogenesis have become known, scientists have experimented with natural and synthetic agents in an effort to control the angiogenic process.

An example of an intracellular protein that is overexpressed in angiogenic endothelial cells is the recently discovered methionine aminopeptidase-2 (MetAP-2). This enzyme is a metalloproteinase that is expressed in both endothelial and nonendothelial cells. However, the expression of MetAP-2 correlates with cell growth; nondividing tissue culture cells lack immunodetectable levels of MetAP-2. It is also an inhibitor of eukaryotic initiation factor 2α (eIF-2α) phosphorylation, which makes it a bifunctional protein. MetAP-2 was discovered when it was found covalently bound to fumagillin, a natural product of fungal origin and a known inhibitor of angiogenesis. Although MetAP-2 is also found in nonendothelial cells, fumagillin has been shown to inhibit endothelial cell cycle progression with some cell type specificity.

Recently, it has been shown that compounds structurally related to fumagillin also bind to MetAP-2. Ovalicin, a sesquiterpene isolated from the fungus Pseudorotium ovalis, has been shown to exhibit antibiotic, antitumour and immunosuppressive activity. Little is known about its molecular mode of action. Ovalicin was found to inhibit angiogenesis with a potency 50 times greater than fumagillin. The IC₅₀ value was estimated at 0.4 nM for ovalicin when 1 nM recombinant human MetAP-2 was used in the assay. It was found that the drug potently inhibits the methionine aminopeptidase activity of MetAP-2. It also has been demonstrated to inhibit cytostatically capillary endothelial cell growth induced by both basic fibroblast growth factor (bFGF) and vascular endothelial cell growth factor (VEGF), and preferentially inhibit endothelial cell growth in tumour vasculature in vivo.

A synthetic analogue of fumagillin, O-(chloroacetylcarbamoyl)fumagillol (TNP-470, also known as AGM-1470), was found to be less toxic and a 50 times more potent inhibitor of angiogenesis than fumagillin. It was also found that the drug potently inhibits the methionine aminopeptidase activity of MetAP-2. The IC₅₀ values were estimated at 1 nM for TNP-470.

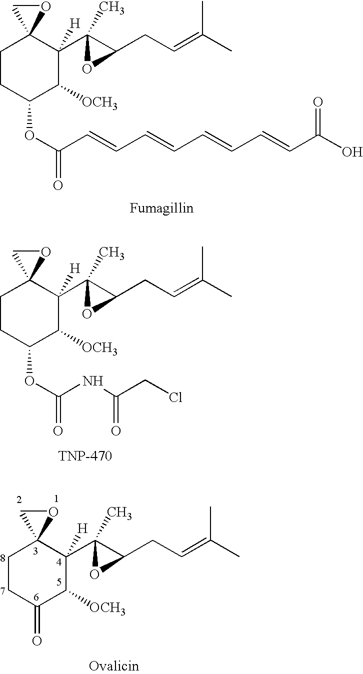

when 1 nM recombinant human MetAP-2 was used in the assay. Similar to ovalicin, TNP-470 cytostatically inhibits the proliferation of endothelial cells in tumour vasculature. In 1992, TNP-470 became the first antiangiogenesis compound to enter clinical trials as an anticancer agent. The molecular mechanism of action of TNP-470 remains unknown, however. It is also not clear whether ovalicin and TNP-470 share the same mechanism of action.

Of the many known extracellular angiogenic protein factors, the most commonly found at sites of tumour growth are basic fibroblast growth factor (bFGF) and vascular endothelial growth factor (VEGF). Both have been shown to induce angiogenesis in vitro and in vivo.

Basic FGF is a widely distributed factor and is present in basement membranes of normal tissues, including normal blood vessels, throughout the body. It is a potent endothelial cell mitogen and chemotactic factor. In addition, it stimulates endothelial cells to produce proteases that can degrade basement membranes. Basic FGF also is a potent stimulator of angiogenesis in in-vivo model systems. It is not specific, however, because it is mitogenic for a wide variety of cell types in addition to endothelial cells.

The angiogenic factor that has the greatest degree of specificity for endothelial cells is vascular endothelial growth factor, or VEGF. VEGF, also known as vascular permeability factor, is substantially overexpressed at both the mRNA and protein levels in a high percentage of malignant animal and human tumours, as well as in many transformed cell lines. VEGF is encoded by a single gene, but exists as four isoforms of 121, 165, 189, and 206 amino acids because of alternative splicing. The two low molecular weight forms, VEGF₁₂₁ and VEGF₁₆₅, are secreted as soluble factors, while the other higher molecular weight forms, VEGF₁₈₉ and VEGF₂₀₆, are secreted but remain bound to extracellular matrix. VEGF₁₂₁ differs from the larger VEGF isoforms in that it is the only VEGF type that does not bind to heparin.

Of the four isoforms, VEGF₁₆₅ is the most abundantly expressed splice variant. It binds to high affinity receptors on the cells surface of vascular endothelial cells. Several types of non-endothelial cells such as human melanoma cells, monocytes, and HeLa cells also express VEGF receptors, but the binding of VEGF₁₆₅ to these receptors does not seem to induce a mitogenic response. In the absence of heparin-like molecules, VEGF₁₆₅ does not bind efficiently to the VEGF receptors of various cell types. VEGF₁₂₁ induces in vascular endothelial cells biological responses that seem to be similar to those induced by VEGF₁₆₅, but does not bind to heparin.

There are various receptors utilized in angiogenesis, but few are as site specific as those for VEGF. There are two high-affinity receptors for VEGF found on endothelial cells: the receptor tyrosine kinases fms-like tyrosine kinase-1 (Flt-1) and kinase domain receptor (KDR), or Flk-1, its murine analogue. Both receptors exert their actions after dimerization precipitated by VEGF and subsequent receptor phosphorylation. Flt-1 has a binding affinity to VEGF of 25 pM and is involved with a later stage of vessel growth. KDR/Flk-1 has a binding affinity of 125 pM and is involved with the differentiation of endothelial cells. Gene disruption experiments in mice show that both receptors are necessary for embryonic development; however, Flk-1/KDR is the primary mitogenic receptor for endothelial cells. It has been shown that both Flt-1 and Flk-1/KDR receptors are overexpressed on the endothelium of tumour vasculature. In contrast, under similar conditions. VEGF receptors are almost undetectable in the vascular endothelium of adjacent tissues.

VEGF is a member of the cysteine-knot family of growth factors. It is a dimer that is held together by two intermolecular disulfide bonds. The NH₂-terminal 110 residues of VEGF₁₆₅ codes for the receptor-binding domain, and the next 55 residues code for the heparin-binding domain. The X-ray crystal structure of the receptor binding domain of VEGF₈₋₁₀₉ has been solved recently, and a high resolution functional map of the binding site for KDR by alanine-scanning mutagenesis has been performed. There are symmetrical binding sites for KDR located at each pole of the VEGF homodimer. Each site contains two functional hot spots composed of binding determinants presented across the subunit interface. The larger hot spot consists of the two most important residues, Ile-46 and Ile-83, and three other residues of lesser importance, Ile-43, Lys-84, and Pro-85. The Arg-82 and His-86 have also been implicated in binding to the KDR/Flk-1 receptor. The Arg-82 to His-86 chain is approximately 4.8× from the Ile-43 to Ile-46 chain. There is one residue of moderate importance from the opposite subunit, Glu-64. The smaller hot spot contains one residue of moderate importance from each subunit, Gln-79 and Phe-17.

The functional requirements for binding and signaling between VEGF₁₋₁₀₉ and KDR have also been analyzed. The data suggests that binding occurs across the VEGF dimer interface. This was shown when a heterodimer containing one molecule of VEGF linked to its homologue PLGF (placenta growth factor) showed a 20-50-fold decrease in binding affinity for KDR receptor. It was proposed that the binding affinity would have been further decreased if PLGF did not retain some of the key binding residues as VEGF, namely Phe-17, Glu-64, Gln-79. and Ile-83. Monomeric VEGF₁₋₁₀₉ was produced by substituting with arginine the two cysteine residues (Cys-51 and Cys-60) that are responsible for the intermolecular disulphides. It was shown that the monomer was properly folded but the binding affinity for KDR was reduced>1000 fold.

Studies performed with VEGF mutants indicated two sites of interaction with Flt-1 that co-localized with the KDR binding determinates. A major site for Flt-1 binding involves the 63-67 region of VEGF, namely Asp-63, Glu-64, and Glu-67. There was a 30-fold reduction in affinity when these residues were replaced with alanine. Mutations at these sites had a minimal affect on KDR binding to VEGF.

Endothelial cells express other receptor tyrosine kinases, notably Tie-1 and Tie-2. The ligand for Tie-1 has not been identified. Two angiopoietic factors, angiopoietin 1 (Ang 1) and angiopoietin 2 (Ang 2), bind to the Tie-2 receptor and play a role in angiogenesis. Ang 1 is ubiquitously expressed and interacts with the Tie-2 receptor expressed on endothelial cells and early hematopoietic cells. Ang 2 is homologous to Ang 1 and competitively inhibits Ang 1 interaction with Tie 2 and disrupts in vivo angiogenesis. Ang 2 appears to be restrictively expressed in areas undergoing vascular remodelling.

There is recent evidence that, from the earliest stages of angiogenesis, endothelial cells destined to become arteries express ephrin-B2, while the cognate receptor, eph B4, is expressed on endothelial cells destined to become veins. Other newly discovered receptors are neurophilin, a receptor for VEGF₁₆₅ which is overexpressed on angiogenic endothelial cells, as are OB-Rβ, the leptin receptor, and the chemokine receptor, CXCR-4. These receptors provide new therapeutic targets for angiogenic sites.

Two proteins, angiostatin, a product of the cleavage of plasminogen, and endostatin, a 20 kDa C-terminal fragment of collagen XVIII, have been discovered to inhibit angiogenesis. Although the mechanism of action is not known, the proteins are intriguing targeting molecules.

Cell adhesion molecules, specifically integrins, also play a part in the angiogenic process. The integrins are a family of transmembrane glycoproteins that are expressed by the cell as αβ heterodimers. There are 16 distinct α subtypes and 8 β subtypes as well as alternative splicing variants of the subunits to form this family. Integrins such as αvβ3, αvβ5, α6, β1, α1β1, α2β1, α4β1, and α5β1 are involved in the regulation of interactions important in angiogenesis. Interactions of αvβ3 with PeCAM-1 on vascular endothelial cells are essential for extravasation. The αvβ3 integrin receptor is highly upregulated on angiogenic endothelial cells and is also found on some tumour cells, such as aggressive melanoma. The natural ligands for αvβ3 include vitronectin and fibronectin, and Cyr61. Preventing the αvβ3 integrin, and in some cases the αvβ5 integrin, from binding to their ligands causes apoptosis in the endothelial cells of newly formed blood vessels.

As with most integrins, αvβ3 has a binding region for proteins which contain the arginine-glycine-aspartic acid (RGD) peptide sequence. It has been shown that blocking the integrin-PeCAM-1 interaction with an RGD peptide inhibits metastatic formation to the vascular endothelium.

Research has shown that the residues flanking the RGD sequence in synthetic peptides have a major effect on the binding affinity and integrin specificity of the peptide. Also, improved binding affinities are claimed to be achieved by conformationally constricting the sequence through cyclization. There have been reports that maintaining the basic portion of the arginine and the acidic portion of the aspartic acid at 14× apart improves binding affinity. Thus, the structure of RGD peptides can be manipulated to bind to particular targets.

The sequence RGD is not the only peptide sequence found to target tumour vasculature. Another sequence is leucine-aspartic acid-valine (LDV). This sequence has been shown to be involved in integrin binding with a similar functional role as RGD. The sequence asparagine-glycine-arginine (NGR) and RPK have also been shown to localize at sites of tumours.

Angiogenic factor expression, in addition to being found at tumour sites, has been linked with the aggressiveness of the tumour, and therefore prognosis of the patient. Tumour angiogenesis has been assessed by staining slices of a tumour and counting the microvascular density (MVD) in the areas of highest neovascularization. Other prognostic indicators include levels of VEGF and bFGF around the tumour site. High MVD has been correlated with poor prognoses in the cases of cervical cancer. High MVD and high expression of the growth factors has been linked with poor survival rates in cases of non-small cell lung cancer, human colorectal cancer, breast cancer, bladder cancer, and many others. Increased receptor expression has also been linked to poor prognoses. Increased expression of the VEGF receptors has been linked with poor survival in invasive breast cancer, pulmonary adenocarcinoma, intestinal-type gastric cancer, and others. Vascular integrin αvβ3 has also been implicated as a prognostic indicator in breast cancer.

The discoveries in the field of angiogenesis have stimulated research in the areas of targeting tumours. Antagonists of angiogenic receptors have been shown to slow down or inhibit tumour growth, as in TNP-470. Examples include antibodies have been developed against Flk-1/KDR and VEGF to prevent VEGF binding and, therefore, angiogenesis; technology that is designed to kill a tumour by causing a blood clot to form in the tumour; soluble VEGF receptors used to inhibit VEGF binding; a variant of VEGF polypeptide that inhibits VEGF binding; and small molecule inhibitors that have been designed for the VEGF receptors. The αvβ3, and sometimes αvβ5, integrin receptors are also a therapeutic targets. Small peptidic molecules, straightchain and cyclic, have been used to inhibit receptor-integrin interactions. There are some peptidomimetic molecules that also show promise at targeting these receptors. In addition, a monoclonal antibody, LM-609, has been shown to block the αvβ3 and halt the proliferation of angiogenic endothelial cells.

These methods of inhibiting angiogenesis suffer from many drawbacks and do not provide the most effective means of targeting angiogenesis for purposes of therapy or diagnostics. Antibodies and large proteins tend to leave a high background of agent in the blood stream for long periods of time, which delays imaging time or keeps a radiotherapeutic circulating in the body.

There have been instances of targeting sites of angiogenesis with chemotherapeutics. However, attaching chemotherapeutics to targeting molecules is problematic because the derivatization of the chemotherapeutic may change its properties. Also, chemotherapeutics have the further drawback of having poor clearance time which causes adverse side effects due to the toxicity of these compounds.

Some attempts have been made to image sites of angiogenesis using magnetic resonance imaging techniques. Polymerized liposomes labelled with gadolinium ions were conjugated to LM-609 and found to localize in angiogenic regions. MRI is a disadvantageous technique for imaging sites of angiogenesis because the amount needed (˜1 g/kg) to get an image often precludes the use of many agents. In addition, there is not a therapeutic counterpart to MRI imaging as there is in nuclear medicine.

There is also one example of targeting the αvβ3 receptor on melanoma tumour cells with a radiolabelled RGD peptide. AngioMab is another monoclonal antibody that has been shown to target tumours when radiolabelled. The peptidic sequence LDV has also been iodinated and thought to target tumours, although it is not clear what is its specific target. However, it has not been previously attempted to label molecules that target sites of angiogenesis with radiopharmaceuticals for the purposes of treatment and imaging. Attacking tumours directly with radiolabelled targeting molecules is problematic in that it is difficult to destroy all of the tumour cells using this technique. Often times tumour cells that are resistant to radiopharmaceuticals will persist and proliferate.

There is therefore a need for a radiolabelled compound that targets sites of angiogenesis. There is a need for such a compound that destroys proliferating endothelial cells at sites of angiogenesis thereby starving the tumour by preventing blood from reaching the tumour. There is a further need for such a compound that images proliferating endothelial cells at sites of angiogenesis for diagnostic purposes.

There is a further need for diagnostic radionuclides that can be used to detect and stage tumours through uptake into the tumour. There is a need for radionuclides useful for therapy that compliment the diagnostic radionuclide, and therefore would have the same co-ordination with the targeting molecule. This partnership would provide an effective method to monitor the progress of the radiotherapy by staging the tumour with the diagnostic before, during, and after treatment.

### SUMMARY OF THE INVENTION

The present invention provides radioactive metal or halogen labeled molecules that target proteins, receptors, or other markers that are upregulated at or on endothelial cells at sites of angiogenesis. The molecules of the present invention include radionuclides useful for imaging or therapy. These molecules are useful for destroying or imaging endothelial cells at sites of angiogenesis.

According to one aspect of the invention, there is provided a compound for imaging angiogenesis or for the treatment of disorders related to angiogenesis of the following formula (I):

A-(B)n-C (I)

wherein

A is a chelator moiety capable of complexing a radionuclide metal or a moiety capable of binding to a halogen;

B is a spacer group;

C is an angiogenesis targeting molecule; and

n is selected from the integers 0 and 1.

According to another aspect of the invention, there is provided a compound comprising a metal chelating moiety and a moiety that binds to sites of angiogenesis.

According to another aspect of the present invention, there is provided a compound for the imaging and treatment of angiogenesis comprising VEGF labeled with an isotope of iodine, technetium, rhenium or an active ester of a metal chelate.

According to yet another aspect of the present invention, there is provided a compound for the imaging and treatment of angiogenesis comprising angiostatin, endostatin, angiopoietin-1, angiopoietin-2, PECAM-1, MMPs, ephrin-B2, osteopoietin, fibronectin, vitronectin, Cyr-61 or pronectin-V labeled with an isotope of iodine, technetium or rhenium.

According to yet another aspect of the present invention, there is provided a kit for preparing a radiopharmaceutical preparation, said kit comprising a sealed vial containing a predetermined quantity of a peptide or molecular reagent according to claim 1 and a sufficient amount of reducing agent to label said reagent with Tc-99m.

According to another aspect of the present invention there is provided a method of treating disorders related to angiogenesis comprising the step of administering to a patient a compound of the following formula (I):

A-(B)n-C (I)

wherein

A is a chelator moiety capable of complexing a radionuclide metal or a moiety capable of binding to a halogen;

B is a spacer group;

C is an angiogenesis targeting molecule; and

n is selected from the integers 0 and 1.

According to another aspect of the present invention there is provided a method of imaging sites of angiogenesis comprising the step of administering to a patient a compound of the following formula (I):

A-(B)n-C (I)

wherein

A is a chelator moiety capable of complexing a radionuclide metal or a moiety capable of binding to a halogen;

B is a spacer group;

C is an angiogenesis targeting molecule; and

n is selected from the integers 0 and 1.

### BRIEF DESCRIPTION OF THE DRAWINGS

FIG. 1 shows the results of a binding assay with HUVECs. The graph shows the dose dependent relationship that certain Re complexes have when competing with 1251-VEGF for binding sites on HUVECs;

FIG. 2 is a graph showing the thymidine incorporation of certain molecules indicating the rate at which endothelial cells proliferate in the presence of certain agents;

FIG. 3 is a plot of % Injected Dose/Gram in turnour of ¹²⁵I-VEGF₁₆₅ in mice against time;

FIG. 4 is a plot showing the tumour:blood ratios in mice of ^{99m}Tc labeled RP511 and RP896 in two separate experiments; and

FIG. 5 is a graph showing the results of a cell adhesion assay with HUVECs and Vitronectin as the natural ligand.

### DETAILED DESCRIPTION OF THE INVENTION

The invention provides targeting molecule-chelator conjugates that, when complexed with a diagnostically or therapeutically useful metal, are useful for imaging or treating sites of angiogenesis. A targeting molecule-chelator conjugate, also referred to as “conjugate”, incorporates the targeting molecule coupled to any linker and any chelator.

In a preferred embodiment of the present invention, the targeting molecule is a peptide that targets the KDR/Flk-1 receptor, as an agonist or antagonist, and incorporates the peptide sequence R-X-K-X-H, or R-X-K-X-H and I-X-X-I arranged in a straightchain, branched, or cyclized, through a linker and flanked by amino acids, amino acid derivatives, or other molecules on each side, in a straight chain, branched, or cyclized fashion.

The targeting molecule can be a peptidomimetic of peptides that target the KDR/Flk-1 receptor.

The targeting molecule can be a peptide that targets the Flt-1 receptor, and incorporates the peptidic sequence D-E-X-X-E in a straightchain, branched, or cyclized fashion. These sequences can be flanked by amino acids, amino acid derivatives, or other molecules, in a straight chain, branched, or cyclized fashion.

The targeting molecule can be a peptidomimetic of peptides that target the Flt-1 receptor. Molecules that target the Tie-1 or the Tie-2 receptor which are upregulated on endothelial cells during angiogenesis can be used as targeting molecules. Angiopoietin-1 and angiopoietin-2 are cleaved by specific proteases to produce peptides that bind to Tie-1 or Tie-2. The following are examples of peptides that are derived from the angiopoietins through cleavage by the proteases TPCK Trypsin or Staphylococcus aureas protease:

GSGYSLK
ATTMMIR
ISSISQPGNDFSTK
DGDNDK
CSQMLTGGWWFDACGPSNLNGXXXX
VGFGNPSGEYWLGNEFVSQLTNQQR
EDGSVDFQR
XXXXTVNNSVLQK
LELQLLEHSLST
LTDVEAQVLNQTTR
EMVEIQQNAVQNQTAVMIEIGTNLLNQTAEQTR
LENYIQDNMK
QYQVQHGSCSYTFLLPEMDNCR
XXXXXXXXXQHGSCSYTFLLPE
NNTQWLMKLE
NYIQD
NMKKE
IQQNAVQNQTAVMIE
IGTNLLNQTAE
QTRKLTD
AQVLNQTTRLE
HSLSTITNKLE
KQILD
KKIVTATVNNSVLQKQQHDLME
AGGGGWTIIQRRE
FQRTWKE
YKVGFGNPSGE
YWLGNE
LNYRIHLKGLTGTAGKISSISQPGND
SQMLTGGWWFD
ACGPSNLNGXXXXXXXXXXKFNGIKWYYWKGSGYSLKATTMMIRPADF

These sequences either alone or flanked by other amino acids, amino acid derivatives, or other molecules arranged as straight chains, branched, or cyclized species, can be radiolabelled or conjugated to a chelator. The targeting molecule can be a peptidomimetic of the peptide. This procedure can also be applied to other proteins including ephrin-B2, VEGF, angiostatin and endostatin.

Targeting molecules of the present invention include radiolabelled peptides and molecules that target upregulated extracellular proteins such as VEGF, angiopoietin or MMPs.

Targeting molecules of the present invention include molecules that target the integrin receptors αvβx (X=3 or 5). The molecules contain the sequence R-X-D or a peptidomimetic thereof and include other amino acids, amino acid derivatives, or other molecules. The peptides can be straightchain, branched, or cyclized, or a combination.

Targeting molecules of the present invention include a molecule of the sequence chelator-X-X-B-X-D-X-X, where B=dimethyl-p-amidinophenylalanine, dimethylarginine, or dimethyllysine.

The targeting molecule can include the sequence dmG-S-C(acm)-G-betaA-betaA-R-G-D-S, amide-capped or as the free acid at the C-terminus.

The targeting molecule-chelator conjugate can include the sequence dmG-S-C(acm)-G-betaA-betaA-R-G-D-S, amide-capped or the free acid at the C-terminus.

The targeting molecule can be a peptidomimetic of R-G-D-S, such as the retro-inverso-dS-dD-G-dR.

Peptides are of the formula dmG-B-C(acm)-D-X-X, where B=dimethyl-p-amidinophenylalanine, dimethylarginine, or dimethyllysine can be used as targeting molecules. The dmG-B-C(acm) portion acts as a chelator for Tc or Re which places the arginine side group and aspartic acid side group about 13× apart. The distance allows better integrin targeting of the peptide than the unlabelled peptide.

Peptides are of the formula dmG-B-C(acm)-Z, where B=dimethyl-p-amidinophenylalanine, dimethylarginine, or dimethyllysine, and Z=homocysteine or 4-aminophenylacetic acid can also be used as targeting molecules according to the present invention.

The targeting molecule of the present invention can also include peptides of the formula dtnB-G-C(acm)-D-X-X where B=dimethyl-p-amidinophenylalanine, dimethylarginine, or dimethyllysine. The distance between the basic portion and the acidic portion is 14× in the metal chelate but 9× in the non-labelled peptide or peptides of the formula dmB-G-C(acm)-Z, where B=dimethyl-p-amidinophenylalanine, dimethylarginine, or dimethyllysine, and Z=homocysteine or 4-aminophenylacetic acid.

Peptides used as targeting molecules can also be of the formula X-(X)n-tripeptide-X-(X)n. Tripeptides such as RGD, NGR, LDV, or RPK localize at sites of angiogenesis. The peptides are arranged in a branched, cyclic, straight chain, or a combination. These peptides can be radiolabelled directly with a halogen, or a chelator can be added to the N or C terminus, or as a sidechain.

The targeting molecule can include proteins, or portions of the proteins that contain the RGD sequence, such as osteopoietin, fibronectin, vitronectin, Cyr61, or pronectin-V (a genetically engineered polymer). These are labelled with a metal, either directly or through a chelator, or a halogen. Targeting molecules of the present invention can also include proteins or portions of proteins such as angiostatin, endostatin, or ephrin-B2, are labelled with a metal, either directly or through a chelator, or a halogen.

Preferred targeting molecules include peptides that have multiple targeting regions and/or multiple labeling or chelating sites.

Radiolabelled targeting molecules that can be used to stage tumours by indicating the degree of vascularization through radiation uptake. Radiolabelled targeting molecules can also be used to detect and treat metastatic tumours.

The compound of the present invention can be of the formula chelator-linker-X-(X)n-tripeptide-X-(X)n and X-(X)n-tripeptide-X-(X)n-linker-chelator where the tripeptide, such as RGD, NGR, LDV, or RPK, is shown to localize at sites of angiogenesis. The peptide can be arranged in a branched, cyclic, straightchain, manner.

In a preferred embodiment of the present invention, the targeting molecule is coupled to the metal chelator of the following formula (I):

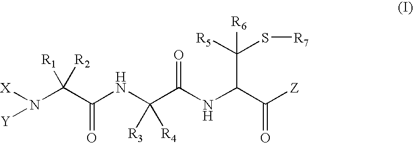

wherein

X is a linear or branched, saturated or unsaturated C₁₋₄ alkyl chain that is optionally interrupted by one or two heteroatoms selected from N, O, and S, and is optionally substituted by one or more substituents selected from halogen, hydroxyl, amino, carboxyl, C₁₋₄alkyl, aryl, and C(O)Z;

Y is H or a substituent defined by X;

X and Y may together form a 5- to 8-membered saturated or unsaturated heterocyclic ring optionally substituted by one or more substituents selected form halogen, hydroxyl, amino, carboxyl, oxo, C₁₋₄alkyl, aryl, and C(O)Z;

R¹ and R⁴ are selected independently from H, carboxyl, C₁₋₄alkyl, C₁₋₄alkyl substituted with a substituent selected from hydroxyl, amino, sulfhydryl, halogen, carboxyl, C₁₋₄alkoxycarbonyl, and aminocarbonyl, an alpha carbon side chain of a D- or L-amino acid other than proline, and C(O)Z;

R⁵ and R⁶ are selected independently from H, carboxyl, amino, C₁₋₄alkyl, C₁₋₄ alkyl substituted by a substituent selected from hydroxyl, carboxyl, amino, and C(O)Z;

R⁷ is selected from H and a sulfur protecting group; and

Z is selected from a hydroxyl, alkoxy, and amino acid residue, and a linking group.

The terms defining the variables R¹-R¹⁰, R^{a}-Rⁿ and X as used hereinabove in formula (I) have the following meanings:

“alkyl” refers to a straight or branched C₁-C₈ chain and includes lower C₁-C₄alkyl;

“alkoxy” refers to straight or branched C₁-C₈ alkoxy and includes lower C₁-C₄alkoxy;

“thiol” refers to a sulfhydryl group that may be substituted with an alkyl group to form a thioether;

“sulfur protecting group” refers to a chemical group that is bonded to a sulfur atom and inhibits oxidation of sulfur and includes groups that are cleaved upon chelation of the metal. Suitable sulfur protecting groups include known alkyl, aryl, acyl, alkanoyl, aryloyl, mercaptoacyl and organothio groups.

“Spacer group” refers to a chemical group that serves to couple the targeting molecule to the chelator while not adversely affecting either the targeting function of the peptide or the metal binding function of the chelator. Suitable spacer groups include alkyl chains; alkyl chains optionally substituted with one or more substituents and in which one or more carbon atoms are optionally replaced with nitrogen, oxygen or sulfur atoms. Other suitable spacer groups include those having the formula A¹-A²-A³ wherein A¹ and A³ are independently selected from N, O and S; and A² includes alkyl optionally substituted with one or more substituents and in which one or more carbon atoms are optionally replaced with nitrogen, oxygen or sulfur atoms; aryl optionally substituted with one or more substituents; and heteroaryl optionally substituted with one or more substituents. Still other suitable spacer groups include amino acids and amino acid chains functionalized with one or more reactive groups for coupling to the glycopeptide and/or chelator. In one embodiment, the spacer group is a peptide of 1 to 5 amino acids and includes, for example, chains of 1 or more synthetic amino acid residues such as β-Alanine residues. In another embodiment, the spacer group is NH-alkyl-NH. The spacer group includes cleavable linkers. Cleavable linkers include esters that can be easily hydrolyzed.

The terms spacer group and linker group have the same meaning.

A “radiopharmaceutical” is a radioactive pharmaceutical or chemical used for the diagnosis or therapeutic treatment of human disease.

“Targeting molecule” refers to a molecule that can selectively deliver a chelated radionuclide or MRI contrasting agent to a desired location in a mammal. Preferred targeting molecules selectively target cellular receptors, transport systems, enzymes, glycoproteins and processes such as fluid pooling. Examples of targeting molecules suitable for coupling to the chelator include, but are not limited to, steroids, proteins, peptides, antibodies, nucleotides and saccharides. Preferred targeting molecules include proteins and peptides, particularly those capable of binding with specificity to cell surface receptors characteristic of a particular pathology. For instance, disease states associated with over-expression of particular protein receptors can be imaged by labeling that protein or a receptor binding fragment thereof coupled to a chelator of invention. Most preferably targeting molecules are peptides capable of specifically binding to target sites and have three or more amino acid residues. The targeting moiety can be synthesized either on a solid support or in solution and is coupled to the next portion of the chelator-targeting moiety conjugates using known chemistry.

The targeting molecules of the present invention target intracellular molecules, such as proteins, that are up-regulated in or are unique to rapidly proliferating endothelial cells at angiogenic sites. The targeting molecules have affinities for intracellular or extra-cellular proteins that are predominantly found at on or around endothelial cells at angiogenic sites, or upregulated extra-cellular angiogenic receptors for proteins such as growth factors, or cell adhesion molecules, such as integrins or selecting, that are found predominantly on endothelial cells at angiogenic sites. The targeting molecules exist as small molecules, peptides, or native proteins.

Chelator conjugates of the invention may be prepared by various methods depending upon the chelator chosen. The peptide portion of the conjugate if present is most conveniently prepared by techniques generally established in the art of peptide synthesis, such as the solid-phase approach. Solid-phase synthesis involves the stepwise addition of amino acid residues to a growing peptide chain that is linked to an insoluble support or matrix, such as polystyrene. The C-terminus residue of the peptide is first anchored to a commercially available support with its amino group protected with an N-protecting agent such as a t-butyloxycarbonyl group (tBoc) or a fluorenylmethoxycarbonyl (FMOC) group. The amino protecting group is removed with suitable deprotecting agents such as TFA in the case of tBOC or piperidine for FMOC and the next amino acid residue (in N-protected form) is added with a coupling agent such as dicyclocarbodiimide (DCC). Upon formation of a peptide bond, the reagents are washed from the support. After addition of the final residue, the peptide is cleaved from the support with a suitable reagent such as trifluoroacetic acid (TFA) or hydrogen fluoride (HF).

Conjugates may fisher incorporate a linking group component that serves to couple the peptide to the chelator while not adversely affecting either the targeting function of the peptide or the metal binding function of the chelator.

The chelator conjugates incorporate a diagnostically useful metal capable of forming a complex. Suitable metals include radionuclides such as ^{99m}Tc, ⁹⁹Tc, ⁶⁴Cu, ⁶⁷Cu, ⁹⁷Ru, 109Pd, ¹⁸⁶Re, ¹⁸⁸Re, ¹¹¹In, ^{113m}In, ¹⁵³Gd, ⁹⁰Y, ¹⁵³Sm, ¹⁶⁶Ho, ¹⁹⁸Au, ¹⁹⁹Au, ⁹⁰Sr, ¹⁰⁵Rh, ²⁰¹Tl, ⁵¹Cr, ⁶⁷Ga, ⁵⁷Co, and ⁶⁰Co. Incorporation of the metal within the conjugate can be achieved by various methods common in the art of coordination chemistry. When the metal is technetium-99m, the following general procedure may be used to form a technetium complex. A peptide-chelator conjugate solution is formed initially by dissolving the conjugate in aqueous alcohol such as ethanol. The solution is then degassed to remove oxygen. Then thiol protecting groups are removed with a suitable reagent, for example with sodium hydroxide and then neutralized with an organic acid such as acetic acid (pH 6.0-6.5). Alternatively, the protecting groups can be removed during the labeling process. In the labeling step, sodium pertechnetate, obtained from a molybdenum generator, is added to a solution of the conjugate with an amount of a reducing agent such as stannous chloride sufficient to reduce technetium and heated. The labeled conjugate may be separated from contaminants ^{99m}TcO₄ and colloidal ^{99m}TcO₂ chromatographically, for example with a C-18 Sep Pak cartridge.

In an alternative method, labeling can be accomplished by a transchelation reaction. The technetium source is a solution of technetium complexed with labile ligands facilitating ligand exchange with the selected chelator. Suitable ligands for transchelation include tartarate, citrate and glucoheptonate. In this instance the preferred reducing reagent is stannous chloride. It will be appreciated that the conjugate may be labeled using the techniques described above, or alternatively the chelator itself may be labeled and subsequently coupled to the peptide to form the conjugate; a process referred to as the “prelabeled ligand” method.

Another approach for labeling conjugates of the present invention involves techniques described in a co-pending U.S. application Ser. No. 08/152,680 filed Nov. 16, 1993, incorporated herein by reference. Briefly, the chelator conjugates are immobilized on a solid-phase support through a linkage that is cleaved upon metal chelation. This is achieved when the chelator is coupled to a functional group of the support by one of the complexing atoms. Preferably, a complexing sulfur atom is coupled to the support which is functionalized with a sulfur protecting group such as maleimide.

When labeled with a diagnostically useful metal, chelator conjugates of the present invention can be used to detect sites of angiogenesis by procedures established in the art of diagnostic imaging. A conjugate labeled with a radionuclide metal such as technetium-99m may be administered to a mammal by intravenous injection in a pharmaceutically acceptable solution such as isotonic saline. The amount of labeled conjugate appropriate for administration is dependent upon the distribution profile of the chosen conjugate in the sense that a rapidly cleared conjugate may be administered in higher doses than one that clears less rapidly. Unit doses acceptable for imaging angiogenesis are in the range of about 5-40 mCi for a 70 kg individual. In vivo distribution and localization is tracked by standard scintigraphic techniques at an appropriate time subsequent to administration; typically between 30 minutes and 180 minutes depending upon the rate of accumulation at the target site with respect to the rate of clearance at non-target tissue.

Radiolabelled targeting molecules with such radionuclides as ¹²³I, ¹²⁵I, ¹³¹I, or ¹⁸F for use as radiopharmaceuticals are typically prepared by either nucleophilic displacement of a suitable leaving group (for example, trifluoromethylsulfonate) using, for example, K¹⁸F by methods known to one skilled in the art, or by an electrophilic substitution of a suitable group (trialkyltin) using for example Na¹²³I in the presence of a suitable oxidant, such as chloramine-T) or related methods known to one skilled in the art. The attachment of halogens, either nonradioactive, radioactive, or other covalently bound elements may also occur through indirect methods such as through the Bolton-Hunter method, stabilized Bolton-Hunter reagents or related methods known to one skilled in the art.

Fumagillol is the targeting portion of fumagillin, and is used as a targeting molecule that, when labelled with a suitable metal, can be used as an imaging agent or a

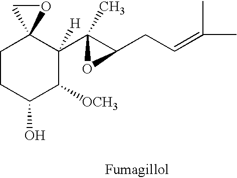

Rhenium is isostructural to technetium. Cold Re is therefore used in place of Tc-99m for in vitro assays. This gives an accurate indication of the conformation of a complex when without having to use Tc-99m. Re-186, or Re-188.

The term “amino acid” derivative includes any molecule, of d or l configuration which contains an amino acid group and a carboxylic acid moiety.

A “peptidomimetic” includes molecules and/or amino acid derivatives that contain the functional groups necessary to provide biological activity similar to that of a biologically active peptide or protein.

Conjugates of the targeting portion of fumagillin, fumagillol may be attached through a linker to a chelator at the C-6 position. The bonds attaching the linker to the C-6 position of fumagillol can include an ester, ether, carbamate, amine, amide, or a carbon-carbon bond.

Conjugates of fumagillin may be attached through the carboxylic acid to a chelator, with or without a linker. The bonds attaching the linker or linker-chelator to fumagillin can include an ester, ether, carbamate, amine, amide, or a carbon-carbon bond.

A chelator may be attached to the fumagillin sidechain at C6 through an amide linkage to the 1,3diaminopropyl linker.

The large proteins vitronectin, fibronectin, PECAM-1, pronectin-V, and osteopoietin all contain the RGD sequence, and would therefore be useful as an αvβ3 targeting agent when radiolabelled.

Pharmaceutical compositions of the above compounds are used to treat patients having disorders related to angiogenesis Vehicles for delivering the compounds of the present invention to target tissues throughout the human body include saline and D5W (5% dextrose and water). Excipients used for the preparation of oral dosage forms of the compounds of the present invention include additives such as a buffer, solubilizer, suspending agent, emulsifying agent, viscosity controlling agent, flavor, lactose filler, antioxidant, preservative or dye. There are preferred excipients for stabilizing peptides for parenteral and other administration. These excipients include serum albumin, glutamic or aspartic acid, phospholipids and fatty acids. The compounds of the present invention may be formulated in solid or semisolid form, for example pills, tablets, creams, ointments, powders, emulsions, gelatin capsules, capsules, suppositories, gels or membranes. Routes of administration include oral, topical, rectal, parenteral (injectable), local, inhalant and epidural administration. The compositions of the invention may also be conjugated to transport molecules to facilitate transport of the molecules. Methods for the preparation of pharmaceutically acceptable compositions which can be administered to patients are known in the art.

Pharmaceutical compositions including the compounds of the present invention can be administered to humans or animals. Dosages to be administered depend on individual patient condition, indication of the drug, physical and chemical stability of the drug, toxicity, the desired effect and on the chosen route of administration (Robert Rakel, ed., Conn's Current Therapy (1995, W. B. Saunders Company, USA)). These pharmaceutical compositions are used to treat cancer.

In an alternate embodiment of the present invention, the compound comprising the targeting molecule, linker and chelator is complexed to a metal that has a therapeutic effect with a non radioactive metal.

In another alternate embodiment of the present invention, the compound includes radiolabelled peptides that have a combined therapeutic effect because of the radionuclide and an antagonistic or inhibitory targeting molecule.

Experimental Section

List of Abbreviations

| Abbreviation | Description |
|---|---|
| acm | acetoamidomethyl |
| AcOH | acetic acid |
| Ar | argon |
| Ala, A | alanine |
| Arg, A | arginine |
| Asn, N | asparagine |
| Asp, D | aspartic acid |
| BetaA | beta-alanine |
| Boc | tert-butyloxycarbonyl |
| BuOH | n-butanol |
| Cys, C | cysteine |
| DDE | 1-(4,4-dimethyl-2,6-dioxocyclohex-1-ylidene)ethyl |
| DIEA | diisopropylethylamine |
| Dimethylgly, dmG | N,N-dimethylglycine |
| Odmab | 4-{N-[1-(4,4-dimethyl-2,6-dioxocyclohexylidene)-3- |
| | methylbutyl]-amino}benzyl ester |
| DMF | N,N-dimethylformamide |
| EDC | 1-[(dimethylamino)propyl]hydrochloride |
| ES-MS | Electron Spray Mass Spectrometry |
| Fmoc | 9-fluorenylmethyloxycarbonyl |
| Gln, Q | glutamine |
| Glu, E | glutamic acid |
| Gly, G | glycine |
| HBTU | 2-(1H-benzotriazol-1-yl)-1,1,3,3-tetramethyl-uronium |
| | hexafluorophosphate |
| His, H | histidine |
| HOBT | 1-hydroxybenzotriazole |
| HPLC | high performance liquid chromatography |
| Ile, I | isoleucine |
| Leu, L | leucine |
| Lys, K | lysine |
| Met, M | methionine |
| mL | millilitre(s) |
| mmol | millimole(s) |
| mol | mole(s) |
| MTT | 4-methoxytrityl |
| NaOH | sodium hydroxide |
| NMP | N-methylpyrrolidone |
| Nle | norleucine |
| Phe, F | phenylalanine |
| Pmc | 2,2,5,7,8-pentamethylchroman-6-sulfonyl |
| PmF | para-amidino-phenylacetic acid |
| Pro, P | proline |
| PyBOP | Benzotriazole-1-yl-oxy-tris-pyrrolidino-phosphonium |
| | hexafluorophosphate |
| Re | rhenium |
| R₁ | retention time |
| sasrin | 2-methoxy-4-alkoxybenzyl alcohol (super acid sensitive resin) |
| Ser, S | serine |
| tb | tert-butyl |
| Tc | technetium |
| TFA | trifluoroacetic acid |
| Thr, T | threonine |
| TFP | 2,3,5,6-tetrafluorophenol |
| Trp, W | tryptophan |
| Trt | trityl |
| Tyr, Y | tyrosine |
| Val, V | valine |
| X | any amino acid of d or l configuration, amino acid derivative, or |
| | other molecule |
| Y-R | protection group R is attached to the peptide chain via the atom, Y, |
| | on the amino acid side chain (Y is N, O or S and R is Acm, Boc, |
| | Mott, t-Bu or Trt) |

Materials

N-methylpyrrolidone, N,N-dimethylformamide, 100 mmol 2-(1H-benzotriazol-1-yl)-1,1,3,3-tetramethyl-uronium hexafluorophosphate/0.5M 1-hydroxybenzotriazole DMF, 2.0M diisopropylethylamine/NMP, dichloromethane and trifluoroacetic acid were purchased from Applied Biosystems Inc. Fumagillin, triethylamine and tert-butyl methyl ether were purchased from Aldrich Chemical Inc. Fmoc amino acid derivatives and Fmoc-Gly sasrin resin was purchased from Bachem Bioscience Inc. All chemicals were used as received.

Instrumentation

Mass spectra (electrospray) were obtained on a Sciex API#3 mass spectrometer in the positive ion detection mode. HPLC analyses and purifications were made on a Beckman System Nouveau Gold chromatographic system with a Waters 4 mm radial pak C-18 column. During analytical HPLC analysis, the mobile phase was changed from 100% 0.1% aqueous trifluoroacetic acid to 100% acetonitrile containing 0.1% trifluoroacetic acid over 20 minutes at a flow rate of 2 mL/min. All HPLC analyses were monitored with a UV detector set at 214 and 254 nm. Solid phase peptide syntheses were performed on an ABI Peptide Synthesizer model 433A using FastMoc chemistry and preloaded Fmoc amino acid sasrin resin. Molecular modeling of the Re complexes was performed using Quanta Charm version 3.3. HPLC analyses of the ^{99m}Tc samples were made on a Beckman System Gold chromatographic system with a Vydac 4.6 mm radial pak C-18 column. The mobile phase was changed from 100% water containing 0.1% trifluoroacetic acid to 70% acetonitrile containing 01% trifluoroacetic acid over 25 minutes at a flow rate of 1 mL/min. The HPLC analyses of the ^{99m}Tc samples were monitored with a UV detector set at 215 nm and a radiometric gamma detector.

### EXAMPLE 1

Synthetic Methods

General Synthesis of Peptides

Peptides of various amino acid sequences were prepared via a solid phase peptide synthesis method on an automated peptide synthesizer using FastMoc 0.25 mmole chemistry. Resin was non-preloaded, preloaded, or amide resin. Fmoc amino acid derivatives were used. Prior to the addition of each amino acid residue to the N-terminus of the peptide chain, the FMOC group was removed with 20% piperidine in NMP. Each Fmoc amino acid residue was activated with 0.50 M 14BTU/HOBt/DMF, in the presence of 2.0M DIEA/NMP. The C-terminus of the completed peptide was attached to the resin via the sasrin linker or a RINK amide linker. The peptidyl resin was washed with dichloromethane and dried under vacuum for 20-24 hours. The peptide was cleaved off the resin by stirring the peptidyl resin in 95% aqueous TFA (Mixture A) or a solution of phenol (0.75 g), ethanedithiol (0.25 mL), thioanisole (0.50 mL), distilled water (0.5 mL), and TFA (10 mL) (Mixture B) for 3-4 hours. The resin was filtered and the filtrate was added dropwise to tert-butyl methyl ether at 0° C. The peptide precipitated out of the ether. The precipitate was collected by centrifugation and dissolved in minimal amount of water. The aqueous peptide solution was lyophilized to yield the product. The product was analyzed by mass spectrometry and by HPLC. The product was purified by HPLC.

Synthesis of Branched Peptides

Peptides contained a lysine residue protected by DDE on the side chain. With the peptide on the resin, the DDE group was removed with 2% hydrazine in NMP. The resin was washed with DCM and dried on a high vacuum for 2 h. The resin was then placed on the synthesizer for further synthesis.

Cyclization of Peptides

Peptidic portions to be cyclized contained a lysine residue with a DDE protecting group on the side chain and a glutamic acid residue with a DMAB protecting group on the side chain. Both of these groups are removed with 2% hvdrazine. The peptides remained on the resin. The resin was swelled in NMP, and PyBOP (4 eq.), HOBT (4 eq), and DIEA (4 eq) was added. The mixture was agitated for 4 days at room temperature under Ar. Cleavage of the peptide was performed by usual methods. The cyclized product was usually present in ˜50% yield. HPLC purification gave a clean product.

Synthesis of Re Starting Materials

The compounds [ReO₂(en)₂]Cl and ReOCl₃(PPh₃)₂ were prepared according to literature methods.

Rhenium Complexation

The chelating peptide exists with the thiol of cysteine protected by an acm group. To remove the acm group, the peptide (0.4 mmol) and mercuric acetate (191 mg, 0.6 mmol) are dissolved in 30% acetic acid (5 mL) and stirred overnight. Hydrogen sulfide is then bubbled through the solution to produce mercuric sulfide as a black precipitate. The mixture is centrifuged, and the supernatant is filtered with a 0.2 m Gelman Acrodisc filter. The filtered sample is lyophilized overnight. The acm deprotected peptide is dissolved in distilled water (8 mL) and [ReO₂en₂]Cl (225 mg, 0.6 mmol) is added to give a light green solution. The pH was adjusted to 7 with 1 N NaOH. The solution is heated at around 80-100° C. for 2 h to produce a red solution. HPLC analysis indicates that the reaction had gone to completion.

Solid Phase Re Complexation

Peptides with an MTT protecting group off the side chain of a cysteine residue were left on the resin. The resin was swelled in ethyl acetate. ReOCl₃(PPh₃)₂ (3 eq) was dissolved in ethyl acetate and the solution was added to the resin. The mixture was agitated for approximately 24 h. The resin was washed with ethyl acetate and DCM. The Re complex was cleaved by cleavage mixture A. The yield of the Re complex depended on the peptide.

Preformed Re Chelate Addition to Peptides

A solution of Re chelated peptide with a free carboxylic acid (1.625 mmol) in acetonitrile:water 4:1 (30 mL) was added to TFP (1.25 g, 8.1 mmol). The pH of this solution was carefully adjusted to pH 2 using 3N hydrochloric acid (˜1.25mL required). To this solution was immediately added EDC hydrochloride (2.5 g, 13 mmol). The pH of the solution was then immediately checked to ensure that it was within the range 4.5 to 5.5. The mixture was vigorously stirred at room temperature for 1 h, during which time the mixture separated into a bilayer. After 30 min progress of the reaction was monitored using thin layer chromatography (silica gel using BuOH:AcOH:H₂O as eluent Rf of product=0.5) to ensure consumption of starting of material. The reaction mixture was poured into a separating funnel and the layers separated. The organic phase was dried over Na₂SO₄, filtered and the solvents removed under reduced pressure. The aqueous was extracted with dichloromethane. This dichloromethane layer was added to the residue obtained from the acetonitrile phase and the organics extracted with brine acidified to pH 3 with hydrochloric acid. The organic phase was dried over NaSO₄ filtered and the solvent removed under reduced pressure. The purity of the TFP ester was checked at this stage by TLC on silica gel (BuOH:AcOH:H₂O). A small amount of TFP impurity at this stage is acceptable. The final yield of the Re-chelate TFP ester was typically ˜50%.

The TFP ester of the chelate in dichloromethane was added to resin-bound peptides containing one or more free amino groups. The mixtures were shaken overnight, over which time the resin turned a red colour. The Re complexes were cleaved of the resin with Mixture A

Synthesis of FMOC-p-Amidino-Phenylalanine

p-Amidino-Phenylalanine was synthesized according to published methods known in the art.

Synthesis of Dimethylamino Amino Acids

The amino acids in there free amine form were stirred with iodomethane in a suitable solvent for 2 h. The amino acid derivatives were purified by column chromatography.

Biological Experimental Data

3H-Thymidine Incorporation Studies

The human umbilical vein endothelial cells (HUVECs, Biowhittaker: passages 2-10) were grown in E-Stim (Becton & Dickinson) or EGM-2 (Biowhittaker) in Collagen-1 coated flasks (B&D). At ˜80% confluence, the cells were collected using trypsin-EDTA (Gibco). The trypsin was neutralized with soybean trypsin-inhibitor (B&D). The cells were washed once with incubation media (consisting of: F-12K (Kaighn's modification) supplemented with 2% fetal bovine serum, 10 mM Hepes and penicillin-streptomycin (all of Gibco). A cell count was performed and viability was determined via trypan-blue exclusion. The cells were resuspended in incubation media at 50 000 viable cells/mL. Ten thousand HUVECs were cultured in 200 μL into each well of a 96-well collagen-1 coated plate. The cells were incubated for 48 h at 37° C. with 5% CO₂. The plates were washed twice with 200 μL of the incubation media. Growth factors and test compounds were dissolved in the incubation media and added to the wells to a final volume of 200 μL. The plates were incubated for another 48 h at 37° C. with 5% CO₂. To each well was added ˜1 μCi ³H-thymidine, diluted in 50 μL of incubation media. The plate was again incubated for 6 h at 37° C. with 5% CO₂. The cells were harvested using a Tomtec cell harvester and 1 μm untreated, glass-fiber filtermat (Skatron), then washed with distilled H₂O. The filtermat was cut and inserted into scintillation counting tubes. About 2-3mL of the scintillation cocktail was added to the tubes, which were mixed and counted for radioactivity associated with the cells.

HUVEC Binding Assays

This method was adapted from a literature method. The HUVECs (BioWhittaker, Walkersville, Md., passages 2 to 10) were cultured at 10,000 viable cells in 0.2 mL/well for 24 h in 96-well collagen-1 coated tissue culture plates at 37° C. with 5% CO₂. The cells were washed twice with 0.2 mL cold PBS. To the cells was added 100 μL of the following dissolved in unsupplemented media (final volume of 200 μL):

Saturation:

¹²⁵I-VEGF₁₆₅ (NEN Life Science Inc., Boston Mass.)

VEGF or media

Competition:

¹²⁵I-VEGF (at or below the Kd value determined from the saturation assays)

VEGF or test compound

The cells were incubated at 4° C. for 2 h. The cells were washed three times with cold water and the cells were harvested with a Skatron cell harvester. The filters were collected and counted for retained radioactivity using a gamma-counter.

Cell Adhesion Assays

HUVECs (BioWhittaker) were cultured on gelatin-coated plates in MCDB 131 media supplemented with 15% fetal bovine serum (FBS), 4 ng/ml basic fibroblast growth factor, 10 ng/ml epidermal growth factor, and 10 U/ml heparin; cells were used in experiments at passages 2-10. The HUVECs are known to express KDR, Flt-1 and αvβ3 receptors.

HUVECs were harvested using 15 mM EDTA pH8.0, washed 3 times, and resuspended in serum-free media. 4×10⁴ cells were preincubated either alone (control) or with test compound for 15 minutes at 37° C., then incubated in 96 well plates coated with known integrin ligand—eg. 5 μg/ml vitronectin (Sigma Aldrich Canada, Oakville, ON) coated 3 hours., 37° C., or RGD-ECM precoated plates (Chemicon International, Temecula, Calif.)—and blocked with 1% bovine serum albumin (BSA). Cells were allowed to adhere for 1-2 hours at 37° C. Unbound cells were removed by washing with phosphate-buffered saline (PBS) and the remaining adherent cells were fixed with 2% paraformaldehyde (30 minutes, room temperature). Adhesion was quantitated by cell staining with crystal violet followed by dye extraction with 10% acetic acid. The absorbance was measured at 595 nm in a microplate reader (Bio-Rad Laboratories, Richmond, Calif.). The sequence RGDS was used as a positive control, and RGES was used as a negative control.

Solid-Phase Receptor Assay

Purified αvβ3 protein (Chemicon) was diluted to 1 μg/ml in coating buffer containing 20 nM Tris pH7.5, 150 mM NaCl, 1 mM MgCl₂, 1 mM CaCl₂, 1 mM MnCl₂. 50 ng per well were added to 96 well Nunc MaxiSorp plates (Canadian Life Technologies, Oakville, ON) and incubated overnight at 4° C. The coated wells were rinsed, blocked with 3% BSA in coating buffer, and incubated with ligand (10 μg/ml vitronectin) for 3 hours at room temperature, in the presence or absence of test competitor compound. Ligand binding was then determined by enzyme-linked immunosorbent assay. For vitronectin binding, wells were incubated with mouse anti-human vitronectin monoclonal antibody (mAb) (5 μg/ml; Chemicon), followed by peroxidase-conjugated rabbit anti-mouse IgG (1:5000 dilution; Jackson ImmunoResearch Laboratories, West Grove, Pa.) and colorimetric detection with the chromogen substrate o-phenylenediamine (OPD) dihydrochloride (Sigma). Absorbances were measured at 490 nm in a microplate reader.

Murine Tumour Xenograft

The murine mammary carcinoma cell line EMT-6 was cultured in Waymouths medium supplemented with 10% FBS. Cells were prepared by trypsinization, washed in serum-free medium, and resuspended to 2.5×10⁶ cells/ml. 200 μl were injected in BALB/c female mice either subcutaneously or intramuscularly on the upper back or in the thigh. Tumour growth was allowed to progress to various time points (4 to 20 days), at which time the mice were sacrificed and the to tumours excised for immunohistological or biodistribution analyses.

Immunohistochemistry

Serial cryostat sections were prepared from frozen tumours and fixed with ice-cold acetone for 10 minutes. The slides were probed using rat anti-mouse CD31 mAb (PharMingen Canada, Mississauga, ON) or mouse anti-Flk1 mAb (Santa Cruz Biotechnology, Santa Cruz, Calif.) and visualized using a biotin/streptavidin-peroxidase system (HISTOSTAIN SP kit; Zymed Laboratories, South San Francisco, Calif.) with AEC chromogen and counterstaining with hematoxylin.

### EXAMPLE 2

KDR/Flk-1 Targeting Peptides

Synthesis

The following peptides were synthesized by the above methods. The identity of the peptides and the Re complexes were confirmed by ES-MS. The compounds were purified by HPLC. A sequence in brackets after an amino acid indicates that the sequence is attached to the side chain of that amino acid. Two asterisks in a sequence indicates that there is a bond between the side groups of those amino acids, and therefore a portion of the peptide is cyclized.

RP704: The Re complex of dmG-S-C(acm)-G.

RP717: The Re complex of dmG-tbG-C(acm)-G

RP543: DmG-S-C(acm)-G-R-I-K-P-H

RP752: RP704-R-I-K-P-H

RP573: DmG-tbG-C(acm)-G-R-I-K-P-H

RP751: RP717-R-I-K-P-H

RP574: DmG-S-C(acm)-G-Q-I-M-R-I-K-P-H

RP757: RP704-G-Q-I-M-R-I-K-P-H

RP584: DmG-K(R-I-K-P-H)-C(acm)-G-I-G-I

RP754:

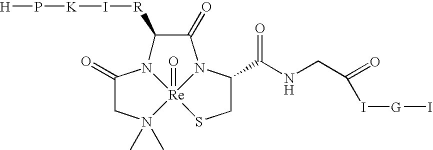

RP590: DmG-K(H-P-K-I-R)-C(acm)-G-K(I-G-G-I)-G

RP800: K(H-P-K-I-R)-G-E*-I-Y-E-I-K*-G

RP805: G-E*-I-E-Y-I-K*-G-R-l-K-P-H

RP761: RP704-G-E*-I-E-Y-I-K*-G-R-I-K-P-H

RP807: G-E*-I-E-Y-I-K*-P-R-I-K-P-H

RP762: RP704-G-E*-I-E-Y-I-K*-P-R-I-K-P-H

RP810: DmG-S-C(acm)-G-R-I-K-P-H-Q-G-Q

RP766: RP704-R-I-K-P-H-Q-G-Q

RP815: H-P-K-I-R-Q-G-Q

RP817: R-I-K-P-H-Q-G-Q

RP821: DmG-S-C(acm)-G-Q-I-Nle-R-I-K-P-H-Q-G-Q

RP759: RP704-Q-I-Nle-R-I-K-P-H-Q-G-Q

RP822: betaA-K(I-Y-E-I)-(P,G,dA)-R-I-K-P-H-Q-G-Q

RP763: RP704-betaA-{(I-Y-E-I)-P,G,dA}-R-I-K-P-H-Q-G-Q

RP824: I-Y-E-I-dK-P-R-I-K-P-H

RP764: I-Y-E-I-dK(RP704)-P-R-I-K-P-H

RP829: betaA-K(I-E-Y-I)-P-P-R-I-K-P-H-Q-G-Q

Re Complex: RP704-betaA-K(I-E-Y-I)-P-P-R-I-K-P-H-Q-G-Q

RP833: DmG-K*-I-E-Y-I-E*-K-P-P-R-I-K-P-H-Q-G-Q

Re Complex: DmG-K*-I-E-Y-I-E*-K(RP704)-P-P-R-I-K-P-H-Q-G-Q

RP835: I-Y-E-I-dK(G-C(acm)-S-dmG)-R-I-K-P-H

RP772: I-Y-E-I-dK(RP704)-R-I-K-P-H

RP837: I-Y-E-I-dK(G-C(acm)-S-dmG)-P-R-I-K-P-H

RP773: I-Y-E-I-dK(RP704)-P-R-I-K-P-H

RP849: betaA-K(I-Y-E-I)-P-R-I-K-P-H-Q-G-Q

RP795: RP704-betaA-K(I-Y-E-I)-P-R-I-K-P-H-Q-G-Q

RP851: betaA-K(I-Y-E-I)-G-R-I-K-P-H-Q-G-Q

RP796: RP704-betaA-K(I-Y-E-I)-G-R-I-K-P-H-Q-G-Q

RP856: betaA-K(I-Y-E-I)-dA-R-I-K-P-H-Q-G-Q

RP797: RP704-betaA-K(I-Y-E-I)-dA-R-I-K-P-H-Q-G-Q

RP876: DmG-S-C(acm)-G-K(P-G-K*-I-dA-Y-I-E*-dmG)-Q-I-Nle-R-I-K-P-H

Re Complex: RP704-K(P-G-K*-I-dA-Y-I-E*-dmG)-Q-I-Nle-R-I-K-P-H

Other Re Complexes:

1) K*-I-E-Y-I-E*-K(RP704)-P-K*-R-I-K-P-H-E*

2) RP704-betaA-K(I-Y-E-I)-dA-K-R-I-K-P-H-E*

Molecular Modelling

The above peptides were modelled and designed to mimic portions of the binding region oft VEGF for the KDR/Flk-1 receptor. The presence of the proline residue in the R-I-K-P-H peptidic fragments allows the histidine residue a conformation similar to that of the native protein.

Cyclization of some of the peptide fragments restricted the conformer to resemble better its counterpart in the native protein. This is particularly the case with the I-X-X-I fragment. In peptides with the R-X-K-X-H and the I-X-X-I fragments, the peptide was designed so that the fragments were about 5×, according to the energy minimized structure by molecular modelling.

Binding Assay

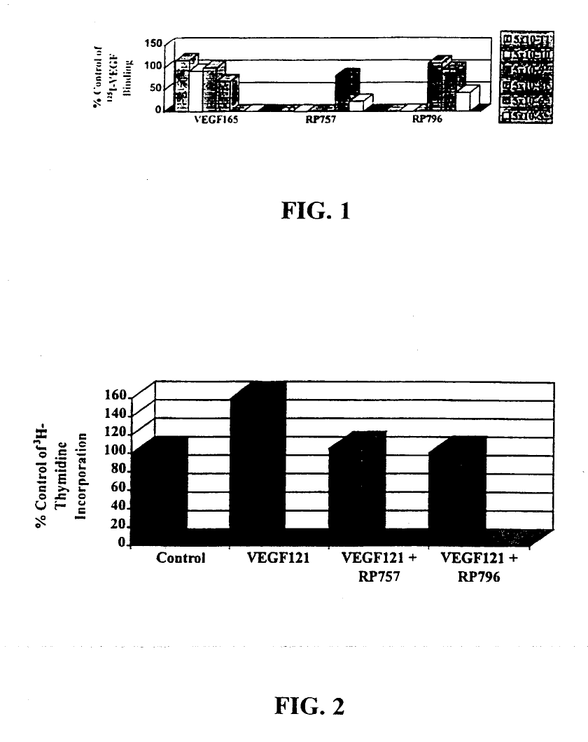

The binding assay shown in FIG. 1 demonstrates the dose dependent relationship that the Re complexes have for competitive binding with ¹²⁵I-VEGF₁₆₅ for the KDR receptor on HUVECs. An example of a Re complex containing the R-X-K-X-H fragment (RP757) and the R-X-K-X-H and I-X-X-I fragments (RP796). These results strongly indicate that the Re complexes of the peptides based on the KDR binding region of VEGF are also binding to the KDR receptor.

Proliferation Assay

The proliferation assay shown in FIG. 2 indicates that the Re complexes of the peptides are binding to the receptor, and that the peptides are acting as antagonists. The control indicates basal growth of the cells. Addition of VEGF₁₂₁ (10⁻¹⁰M) caused the cells to proliferate to about 150% of the basal growth. Addition of RP757 and RP796 (each at 10⁻⁵M) to the cells and VEGF₁₂₁ inhibited the proliferation of the cells.

### EXAMPLE 3

Peptides Targeting Flt-1

Peptides

The following peptides and Re complexes were synthesized and characterized according to the above methods:

RP537: DmG-S-C(acm)-G-D-E-G-L-E

RP753: RP704-D-E-G-L-E

RP571: DmG-tbG-C(acm)-G-D-E-G-L-E

RP750: RP717-D-E-G-L-E

Binding Assay

These Re complexes are tested for binding affinity with HUVECs against ¹²⁵I-VEGF₁₆₅ the natural ligand for the Flt-1 receptor. Molecules with good binding affinity are selected to target Fit-1 receptor.

In Vivo

The peptides are labelled with Tc-99m and examined for localization in tumours via a biodistribution in murine tumour models. Molecules that are retained in tumours are selected as targeting molecules.

### EXAMPLE 4

Dimethyl-Gly-Ser-Cys(acm)-NH(CH2)₃NH-Fumagillin (RP519)

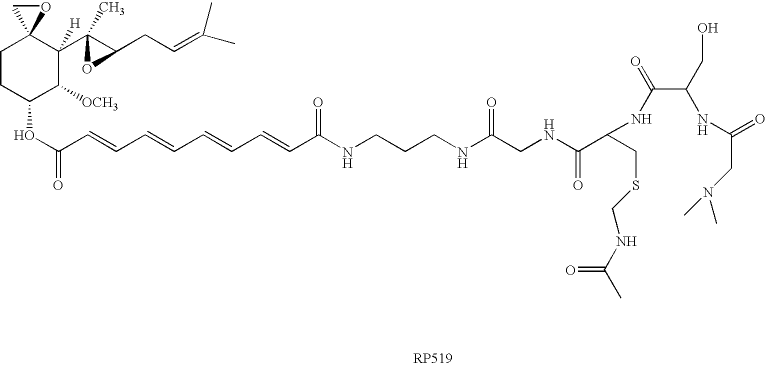

Synthesis of RP492

The dmG-Ser(Bu)-Cys(Acm)-Gly-NH-(CH₂)₃-NH2 (RP492) portion of RP519 was prepared as a single peptide chain by solid phase protein synthesis (SPPS) as detailed above. In this case, the 0.25 mmol chemistry required preloaded 1,3-diaminopropyltrityl resin (0.40 mmol/g, 0.25 mmol, 625 mg), and 1 mmol of each representative amino acid derivative, dmG, Fmoc-Ser(tBu), Fmoc-Cys(Acm), and Fmoc-Gly. The peptide was cleaved with cleavage Mixture B. The lyophilized product gave an off-white powder. Crude product (408 mg, 85.5%): HPLC (0 to 100% ACN in 20 minutes) R₁ 4.83 minutes, ES-MS (C₁₈H₃₅N₇O₆S, m.w. 477.24) m/z 478.23 (M⁺).

Synthesis of RP519

RP492 (5 mg, 0.012 mmol) and fumagillin (2.5 mg, 0.006 mmol) were dissolved in 500 μL DMF. To this solution was added 0.45 M HBTU/HOBT in DMF (18 μL, 0.008 mmol), and DIEA (4 μL, 0.022 mmol). The solution was stirred under Ar at room temperature for 24 hours. Analytical HPLC showed a new peak at 13.3 minutes. The sample was HPLC purified ESMS (MW=917 g/mol) m/z=918 [M⁺].

Tc-99m Labelling of RP519

RP519 (200 μg) was dissolved in saline (200 μL). 99mTc-pertechnetate was added (10 mCi) followed by stannous gluconate (100 μL). The solution was swirled well and left to stand at room temperature for 60 min. HPLC analysis indicated that the radiolabelled product appeared at 24 min with a radiochemical purity of >30%. The product was with 50% EtOH/PBS from a C-18 Sep Pak column to give a pure product.

Synthesis of Fumagillol

Fumagillol is synthesized according to the method by Landquist. Fumagillin (10 mg), methanol (1 mL), and 40% aqueous sodium hydroxide (100 μL) are boiled under reflux for 1.5 hours, then cooled. The sodium octatetraene-1: 8-dicarboxylate is filtered off. The filtrate is evaporated under reduced pressure at 30° C. and the residue is dissolved in water and extracted with ether (3×1 mL). Evaporation of the dried (Na₂SO₄) extract gives a syrupy product.

Synthesis of Fumagillol-Glycine-FMOC

The synthesis is performed according to Crews et al. Fluorenylmethoxycarbonyl-Gly-OH (5.3 mg, 18 μmol) and oxalylchloride (3.0 μl, 24.0 μmol) are combined in CH₂C₂ (500 μl) and treated with a catalytic amount dimethylformamide (0.3 μl, 1.0 μmol). The resulting mixture is stirred at room temperature under a nitrogen atmosphere for 3 h. The solvent is removed and the residue is stirred under vacuo for 0.5 h. In another flask, fumagillol (1.54 mg, 5.5 μmol) and 4-dimethylaminopyridine (2.6 mg, 16 μmol) are combined in CH₂Cl₂ (200 μl). A CH₂Cl₂ (200 μl) solution of acid chloride above is added to this mixture. After 3 h, the solvent is removed and the product is purified by flash column chromatography (silica gel, 1:1 hexanes:EtOAc) to give Fmoc-glycine-tethered fumagillol (1.2 mg, 60% yield based on recovered starting material.

The Fmoc-glycine-fumagillol (1.6 mg, 2.9 μmol) is stirred in 20% piperidine-dimethylformamide (200 μl) for 20 min. The solvent is removed under vacuo, and the product is purified by flash column chromatography (silica gel, 95:5 CH₂Cl₂/MeOH) to give glycine-fumagillol (0.9 mg, 93.1%) Synthesis O-(1,3 Diaminopropane)Fumagillol

A 1,3 diaminopropane is added to the C-6 position of fumagillol through a carbamate bond via a method derived from Ingber et al.

Synthesis of RP414-Glycine-Fumagillol and the Re Complex

The TFP ester of RP414 is made by adding TFP (5 eq.) with EDC (10 eq.) as a coupling agent in 9 ACN:1 H₂O at a pH of 4 and room temperature. It is added to the amino group of fumagillol-glycine or O-(1,3-diaminopropane)fumagillol. The Re complex is made by one of the above syntheses.

Biological Studies

The ^{99m}Tc complexes of fumagillin and fumagillol conjugates are tested for activity in cellular proliferation assays with HUVECs and tested for tumour uptake in murine tumour model in vivo studies. Molecules that result in inhibition of cellular proliferation of endothelial cells are selected as targeting agents.

### EXAMPLE 5

Iodinated VEGF₁₆₅

Synthesis of ¹²³I-VEGF₁₆

The VEGF is iodinated according to methods known in the art.

Biodistribution of ¹²⁵I-VEGF₁₆₅

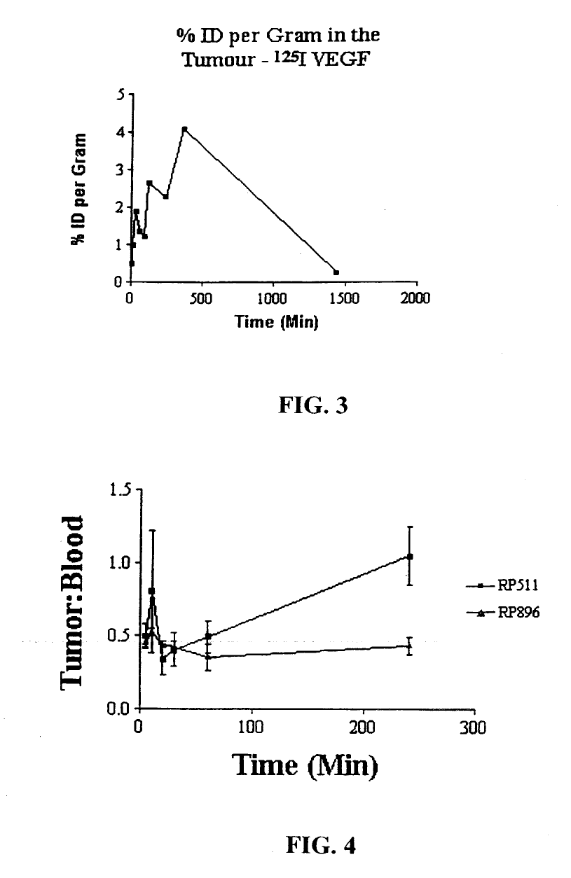

FIG. 3 sets out a graph demonstrating the accumulation of VEGF₁₆₅ in a mouse tumour.

The murine breast carcinoma EMT-6 cell line was injected subcutaneously into the thigh of female Balb/C mice. At twelve days, ¹²⁵I-VEGF₁₆₅ was injected and the distribution was determined at various time points. Excessive vascularization was seen in this tumour model in a previous experiment which included excising, slicing, and staining the tumours at 12 days with the vessel stain CD31.

As shown in FIG. 3, tumour uptake of ¹²⁵I-VEGF₁₆₅ peaked at 6 h with a % ID/g of 4.1. The ¹²⁵I-VEGF₁₆₅ was internalized and degraded in the cells. Subsequent in vitro experiments confirmed this hypothesis (vide supra).

Internalization/Efflux of ¹²⁵I-VEGF₁₆₅

¹²⁵I-VEGF₁₆₅ was rapidly internalized into HUVECs with a greater than 75% of the internalized protein at 2 h and slowly decreased to 60% at 6 h. Separation of effluxed products indicated that the majority of the activity was greater than 10 kDa, which suggests that the intracellular degradation products of ¹²⁵I-VEGF₁₆₅ produced predominantly large peptidic fragments.

These data prove that the ¹²⁵I-VEGF₁₆₅ is targeting the endothelial cells. The high tumour uptake of the protein indicates its utility as a radioimaging or radiotherapeutic agent when labelled with a suitable metal.

### EXAMPLE 6

Tc-99m Labelled VEGF₁₆₅

The VEGF₁₆₅ is labeled directly with Tc-99m by methods known in the art. Several methods of direct labeling of proteins have been reported; some proteins which have been radiolabelled by these methods include human serum albumin, fibrinogen, various enzymes, cells, antibodies and many others. The reaction is carried out in a one step synthesis by the addition of stannous chloride to a protein solution at acidic pH. An alternate direct labeling method is to “pretin” the protein with SnCl₂ for up to 21 hours at room temperature. Sodium pertechnetate is then added, in the absence of air. A modified version of the direct labelling approach involves the pre-attachment of a chelating group to the protein followed by the addition of reduced technetium-99m. An alternate methodology is to pre-label the chelator with Tc-99m and then bind the preformed Tc-99m complex directly to the protein.

Unlabeled VEGF has the same biological activity as VEGF when labeled with ^{99m}Tc.

Other proteins that are labelled by this approach with Tc-99m, Re-186, or Re-188 include: angiopoietin-1 and -2, angiostatin, endostatin, Cyr61, pronectin-V, cell adhesion molecules such as vitronectin and fibronectin, and MMPs.

### EXAMPLE 7

Binding to αvβ3 Receptor by Peptides Having the Sequence DmG-S-C(acm)-G-betaA-betaA-R-G-D-S-NH₂ (RP511)

Peptides

The peptide RP511 and its Re complex were synthesized according to the above methods. A negative control, dmG-S-C(acm)-G-betaA-betaA-R-G-E-S-NH₂ (RP896) and its Re complex were also synthesized. The R-G-E-S sequence does not bind to the αvβ3 receptor.

Cell Adhesion Assay

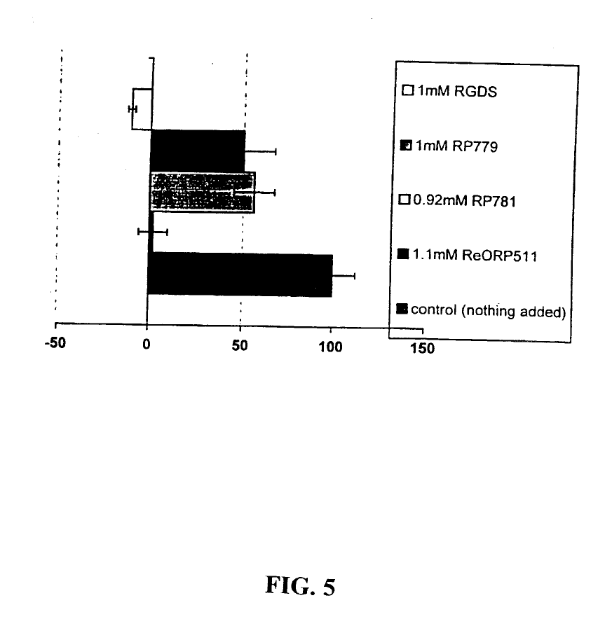

The cell adhesion assay tests the ability of a peptide to block vitronectin or fibronectin from binding to the αvβ3 receptor. The positive control, R-G-D-S, shows 100% inhibition of binding. FIG. 5 shows that the re complex of RP511, ReORP511, exhibits almost 100% inhibtion of binding. This is remarkable considering the bulky Tc-99m chelate added to the N-terminus. The results indicate that ReORP511 is targeting the αvβ3 receptor on endothelial cells.

Biodistribution

FIG. 4 shows the tumour to blood ratios of Tc-99m labelled RP511 and RP896 in the EMT-6 tumour model.

The biodistributions were performed on 8 day EMT-6 tumours injected sub-cutaneously in the upper back with Tc-99m labelled RP511, and Tc-99m labelled RP896 in a separate experiment. The EMT-6 cell line is not known to express the αvβ3 receptor. FIG. 5 clearly shows that there is retention of RP511, but not of RP896. The tumour:muscle ratio of Tc-99m labelled RP511 is 8 at 4 h.

The in vitro and in vivo results clearly indicate that radiolabelled RP511 is targeting the αvβ3 receptors of the tumour vasculature, and the agent has great potential as a radiopharmaceutical to detect tumours and metastases, to stage, and treat tumours.

### EXAMPLE 8

Binding to αvβ3 Receptor by Compounds Having the Sequence R-(d or l) K(chelator)-D-S

Peptides

The following peptides and Re complexes were synthesized according to the above methods.

RP852: R-dK(G-C(acm)-S-dmG)-D-S

RP779: R-dK(RP704)-D-S

RP854: R-K(G-C(acm)-S-dmG)-D-S

RP781: R-K(RP704)-D-S

Cell Adhesion Assay

The Re complexes are shown to have some affinity for the αvβ3 receptor in the cell adhesion assay (FIG. 5). This molecule mimics the R-G-D motif. It is remarkable that affinity is seen for the αvβ3 receptor considering the bulky Re chelate off the lysine residue where traditionally there is no side group. FIG. 5 sets out the results of a cell adhesion assay with HUVECs and vitronectin as the natural ligand. The cell adhesion assay gives an indication of how vitronectin is binding to the HUVECs through the αvβ3 receptor. The control shows the amount of HUVECs that bind to vitronectin coated on a plate when there is no interference. Addition of RGDS eliminated the binding because it is blocking the receptors. ReORP511 also inhibits the binding, because it is also blocking the receptors. RP779 and RP781 show some affinity for the αvβ3 receptor.

### EXAMPLE 9

Binding to αvβ3 Receptor by Peptides Having the Sequence DmG-B-C(acm)-ZX and Re Complexes

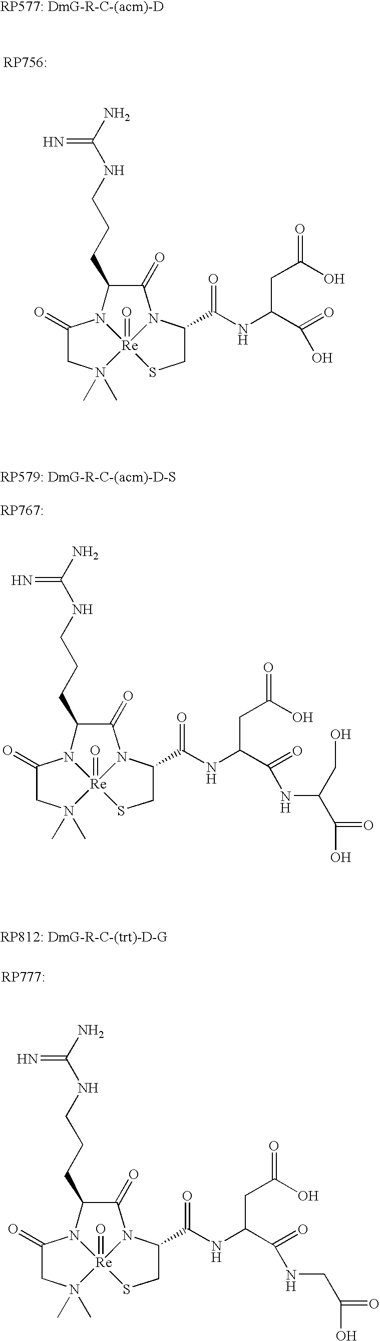

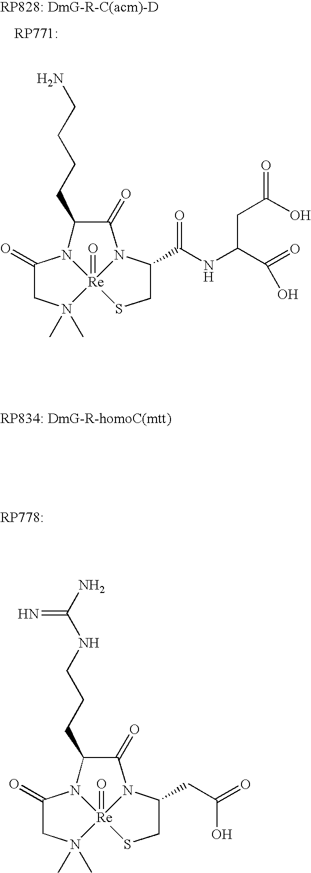

Molecular Modelling

The above peptides, before complexed with Re, had a distance between the basic and acidic functional groups of about 9 Å by molecular modelling. The ideal distance for conformationally restricted peptides is said to be 14 Å. After complexation of Re, the distance is 13 Å. The C(acm) group acts as a linker between the basic and acidic side groups once it is complexed to the metal, thereby mimicking the glycine in the RGD sequence. Similar results are expected with Tc-99m, as the two metals are isostructural. These peptides give higher specific activity at αvβ3 sites because the labelled peptides will have a higher binding affinity than the unlabelled.

Biological Studies

The Re complexes are tested in cell adhesion assays, and the Tc-99m are tested for tumour uptake in biodistributions with murine tumour models.

### EXAMPLE 10

Binding to αvβ3 Receptor by Peptides Having the Sequence DmB-G-C(acm)-D-X and Re Complexes

Synthesis

The following peptides and Re complexes were synthesized and characterized according to the above methods:

DmR-G-C(acm)-D-S

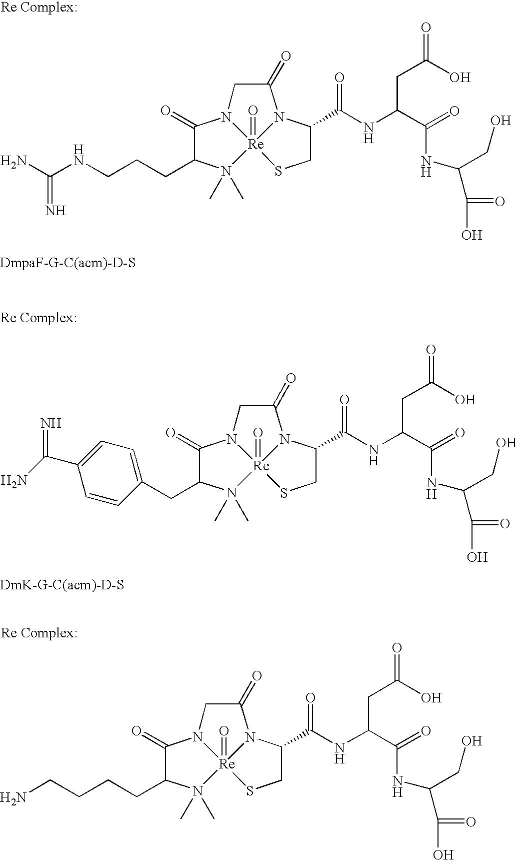

Molecular Modelling

The minimized structures of the Re complexes show that the basic groups and the acidic groups are the ideal distance of Å. The uncomplexed peptide shows a distance of 9 Å. This indicates that the complexed peptide has higher affinity, and therefore should give higher specific affinity at αvβ3 sites.

Biological Studies

The Re complexes are tested in cell adhesion assays, and the Tc-99m are tested for tumour uptake in biodistributions with murine tumour models.

### EXAMPLE 11

Binding to αvβ3 Receptor by Peptides Having the Sequence DmB-G-C(acm)-4-Amino-Phenylacetic Acid and Re Complexes

Synthesis

The following peptides and Re complexes were synthesized and characterized according to the above methods:

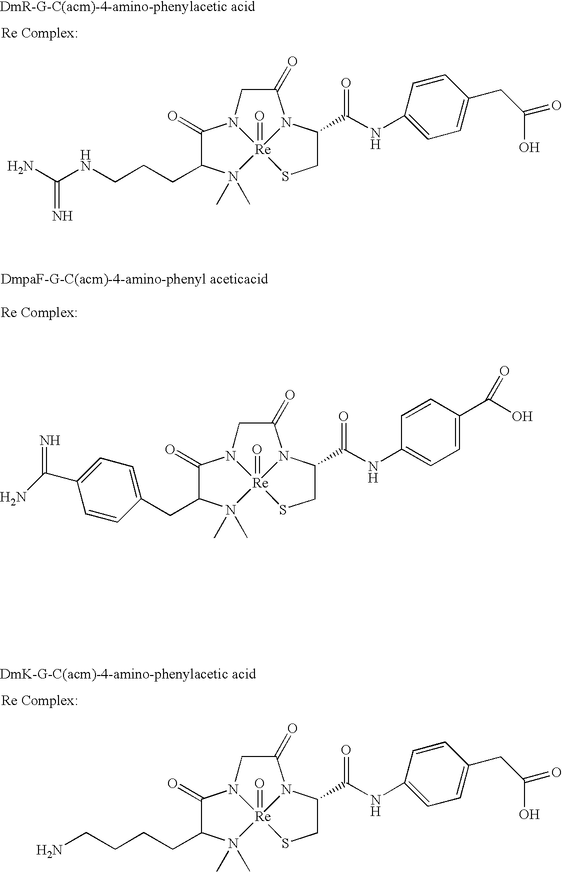

Molecular Modelling

The minimized structures of the Re complexes show that the basic groups and the acidic groups are the ideal distance of Å. The uncomplexed peptide shows a distance of 9 Å. This indicates that the complexed peptide has higher affinity, and therefore should give higher specific affinity at αvβ3 sites.

Biological Studies

The Re complexes are tested in cell adhesion assays, and the Tc-99m are tested for tumour uptake in biodistributions with murine tumour models.

### EXAMPLE 12

Binding to αvβ3 Receptor by Peptides Having the Sequence DmB-G-C(acm)-Homocysteine and Re Complex

The 4-aminophenylacetic acid of the peptides and Re complexes of Example 11 is replaced by homocysteine.

### EXAMPLE 13

Binding to αvβ3 Receptor by Peptides Having the Sequence DmG-S-C(acm)-G-betaA-betaA-dS-dD-G-dR-NH₂and its Re Complex

Synthesis

The following peptide and Re complex were synthesized by the above synthetic techniques.

RP863: DmG-S-C9acm)-G-betaA-beta A-dS-dD-G-dR

Re complex: Rp704-betaA-betaA-dS-dD-G-dR

Cell Adhesion Assay

The Re complex of this retro-inverso mimic of Rp511 is tested in cell adhesion assays, and the Tc-99m labeled peptide is tested for tumour uptake in biodistributions with murine tumour models. The retro-inverso nature of the peptide allows greater in-vivo stability than R-G-D-S.

### EXAMPLE 14

Targeting Sites of Angiogenesis by Peptides Having the Sequence DnG-S-C(acm)G-beta-beta-L-D-V and its Re Complex

The following Re complexes were synthesized according to the above methods.

RP866: DmG-S-C(acm)-G-betaA-betaA-L-D-V

Re complex: RP704-betaA-betaA-L-D-V

Cell Adhesion Assay

The 99m labeled peptide is tested for tumour uptake in biodistributions with murine tumour models.

### EXAMPLE 15

Binding to αvβ3 Receptor by Peptides Having the Sequence DmG-S-C(acm)-G-[linker-K(R-G-D-S)-linker-K(R-G-D-S)]n

Synthesis

This multiple targeting molecule peptide-chelator conjugate and Re complex is made by the above synthetic techniques.

Biological Assays

The Tc-99m labelled peptide is tested for tumour uptake in murine tumour models. There is more uptake because the multiple binding regions provides a better chance for the peptide to bind to the αvβ3 receptors.

### EXAMPLE 16

Deriving Peptides from Proteins that Target Angiogenic Sites Through Enzyme Cleavage:

Angiopoietin-1 and -2

Angiopoietin-1 and angiopoietin-2 are cleaved by specific proteases to produce peptides that bind to Tie-1 or Tie-2. The following are examples of peptides that are derived from the angiopoietins through cleavage by the proteases TPCK Trypsin or Staphylococcus aureas protease:

GSGYSLK
ATTMMIR
ISSISQPGNDFSTK
DGDNDK
CSQMLTGGWWFDACGPSNLNGXXXX
VGFGNPSGEYWLGNEFVSQLTNQQR
EDGSVDFQR
XXXXTVNNSVLQK
LELQLLEHSLST
LTDVEAQVLNQTTR
EMVEIQQNAVQNQTAVMIEIGTNLLNQTAEQTR
LENYIQDNMK
QYQVQHGSCSYTFLLPEMDNCR
XXXXXXXXXQHGSCSYTFLLPE
NNTQWLMKLE
NYIQD
NMKKE
IQQNAVQNQTAVMIE
IGTNLLNQTAE
QTRKLTD
AQVLNQTTRLE
HSLSTTNKLE
KQILD
KKIVTATVNNSVLQKQQHDLME
AGGGGWITIIQRRE
FQRTWKE
YKVGFGNPSGE
YWLGNE
LNYRIHLKGLTGTAGKISSISQPGND
SQMLTGGWWFD
ACGPSNLNGXXXXXXXXXXKFNGIKWYYWKGSGYSLKATTMMIRPADF

These peptides are radiolabelled directly or through the preformed chelate method. The radiolabelled peptides are tested for tumour uptake in biodistribution studies in murine tumour models. Peptides that show uptake are further developed as diagnostic and therapeutic radiopharmaceuticals.

The same method can be used on: VEGF, angiostatin, endostatin, Cyr61, pronectin-V, cell adhesion molecules such as vitronectin and fibronectin, ephrin B-2and MMPs. These peptides can be categorized according to their size using size exclusion columns.

Although the invention has been described with preferred embodiments, it is to be understood that modifications may be resorted to as will be apparent to those skilled in the art. Such modifications and variations are to be considered within the purview and scope of the present invention.

References

1. Folkman, J. Angiogenesis in cancer, vascular, rheumatoid, and other disease. Nature Med. 1, 27-31 (1995).

2. Hanahan, D. Signaling vascular morphogenesis and maintenance. Science 277, 48-50 (1997).

3. Risau, W. Mechanisms of angiogenesis. Nature 386, 671-674 (1997).

4. Li, X., and Chang, Y. -H., (1995). Biochim. Biophys. Acta 1260, 333-336.

5. Ray, M. K., Datta, B., Chakraborty, A., Chattopadhyay, A., Meza-Keuthen, S., and Gupta, N. K. (1992). Proc. Natl. Acad. Sci. USA 89, 539-543.

6. Hanson, F. R., and Eble, T. E. (1949). An antiphage agent isolated from Aspergillus sp. J. Bacteriol. 58, 527-529.

7. McCowen, M., Callender, M., and Lawlis, J. (1951). Fumagillin (H-3), a new antibiotic with amebicidal properties. Science 113, 202-203.

8. Sin, N., Meng, L., Wang, M. Q. W., Wen, J. J., Bornmann, W. G., and Crewa, C. M. (1997). The antiangiogenic agent fumagillin covalently binds and inhibits the methionine aminopeptidase, MetAP-2. Proc. Natl. Acad. Sci. USA 94, 6099-6103.

9. Kusaka. M., Sudo, K., Matsutani, E., Kazai, Y., Marul, S., Fujita, T., Ingber, D., and Folkman. J. (1994). Br. J Cancer 69, 212-216.

10. Sigg, H. P., and Weber, H. P. (1968). Isolation and structure elucidation of ovalicin. Helv. Chim. Acta 51, 1395-1408.

11. Bollinger, P., Sigg, H. P., and Weber, H. P. (1973). Structure of ovalicin. Helv. Chim. Acta 56. 819-830.

12. Zimmerman, W. A., and Hartman, G. R., (1981). On the mode of action of the immunosuppressive sesquiterpene ovalicin. Eur. J. Biochem. 118, 143-150.

13. Borel, J. F., Lazary, S., and Staehelin, H. (1974). Immunosuppressive effects of ovalicin-semicarbazone. Agents Actions 4.357-363.

14. Corey, E. J., Guzman-Perez, A., and Noe, M. C. (1994). Enantioselective synthesis of (−)-ovalicin, a potent inhibitor of angiogenesis, using substrate-enhanced catalytic asymmetric hydroxylation. J. Am. Chem. Soc. 116, 12109-12110.

15. Griffith, E. C., Su, Z., Turk. B. E., Chen, S., Chang, Y -H., Wu, Z., Biemann, K., and Liu, J. O. (1997). Methionine aminopeptidase (type 2) is the common target for angiogenesis inhibitors AGM-1470 and ovalicin. Chemistry & Biology 4(6), 461-471.

16. Toi, M; Takayanagi, T., Souma, R., and Tominaga, T. (1994). Inhibition of vascular endothelial growth factor induced cell growth by an angiogenesis inhibitor AGM-1470 in capillary endothelial cells. Oncology Reports 1, 423-426.

17. Yamamoto, T., Sudo, K., and Fujita, T. (1994) significant inhibition of endothelial growth in tumour vasculature by an angiogenesis inhibitor TNP-470 (AGM-1470). Anticancer Res. 14, 1-4.

18. Ingber, D., et al. And Folkman, J. (1990). Synthetic analogues of fumagillin that inhibit angiogenesis and suppress tumour growth. Nature 348, 555-557.

19. Dvorak, H. F., Detmar, M., Claffey, K. P., Nagy, J. A., van de Water, L, and Senger, D. R., Vascular permeability factor/vascular endothelial growth factor: an important mediator of angiogenesis in malignancy and inflammation. Int. Arch. Allerg. Immunol., 107, 233-235 (1995).

20. Senger, D R, Galli, S J, Dvorak, H F: A highly conserved vascular permeability factor that promotes accumulation of ascites fluid. Science, 1983, 219: 983-985.

21. Senger, D R, Perruzzi, C A., Feder, J, Dvorak, H F: A highly conserved vascular permeability factor secreted by a variety of human and rodent tumour cell lines. Cancer Res. 1986, 46: 5629-5632.

22. Tischer, E., Mitchell, R., Hartman, T., Silva, M, Gospodarowicz D., Fiddes, J C, Abraham J A: The human gene for vascular endothelial growth factor. Multiple protein forms are encoded through alternative exon splicing. J Biol Chem 1991, 266: 11947-11954.

23. Houck, K A, Leung, D W, Rowland, A M, Winer, J., and Ferrara, N. (1992) J Biol Chem 267, 26031-26037.

24. Houck, K A, Ferrara, N, Winer, J., Cachianes, G., Li, B., and Leung, O W (1991) Mol. Endocrinol. 5, 1806-1814.

25. Vaisman, N., Gospodarowicz, D., and Neufeld, G., (1990) J Biol. Chem 265, 19461-19466.

26. Plouet, J., and Moukadiri, H., (1990) J. Biol. Chem. 265 22071-22074.

27. Gitay-Goren, H., Soker, S., Vlodavsky, I., and Neufeld, G. (1992) J Biol Chem. 267, 6093-6098.

28. Gitay-Goren, H., Halaban, R., and Neufeld, G., (1993) Biochem. Biophys. Res. Commun., 190, 702-709.

29. De Vries, C., Escobedo, J. A., Ueno, H., Houck, K. A., Ferrara, N., and Williams, L. T., The fms-like tyrosine kinase, a receptor for vascular endothelial growth factor. Science, 255, 989-991 (1992).

30. Fong, G -H., Rossant, J., Gertenstein, M. m and Breitman, M. I. (1995) Nature 376, 66-70.

31. Millauer, B., Wizigmann-Voss, S., Schnurch, H., Martinez, R., Moller, N. P., Risau, W., and Ulrich, A., High affinity binding and developmental expression suggest Flk-1 as a major regulator of vasculogenesis and angiogenesis. Cell, 72, 835-846 (1993).

32. Shalaby, F., Rossant, J., Yamaguchi, T. P., Gertenstein, M., Wu, X -F., Breitman, M. L., and Schuh, A. C. (1995). Nature 376, 62-66.

33. Brown, L. F., Berse, B., Jackman, R. W., Tongazzi, K., Manseau, E. J., Senger, D. R., and Dvorak, H. F., Expression of vascular permeability factor (vascular endothelial factor) and its receptors in adenocarcinomas of the gastrointestinal tract. Cancer Res., 53, 4727-4735 (1993).

34. Plate, K. H., Breier, G., Millauer, B., Ulrich, A., and Risau, W., Up-regulation of vascular endothelial growth factor and its cognate receptors in a rat glioma model of tumour angiogenesis. Cancer Res., 53, 5822-5827 (1993).

35. Sun, P. D., and Davies, D. R. (1995) Annu. Rev. Biophys. Biomol. Struct. 24, 269-291.

36. Muller, Y. A., Li, B., Christinger, H. W., Wells, J. A., Cunningham, B. C., and De Vos, A. M. (1 997) Proc Natl Acad. Sci. U.S.A. 94, 7192-7197.

37. Fuh, G., Li, B., Crowley, C., Cunningham, B., and Wells, J., Requirements for binding and signaling of the kinase domain receptor for vascular endothelial growth factor. J Biol Chem 1998, 273, 11197-11204.

38. Keyt, B. A., Nguyen, H. V., Berleau, L. T., Duarte, C. M., Park, J., Chen, H., and Ferrara, N. 1996 Identification of Vascular endothelial growth factor determinants for binding KDR and Flt-1 receptors. J. Biol. Chem. 271: 5638-5646.

39. Suri, C., Jones, P. F., Patan, S., Bartunkova, S., Maisonpierre, P. C., Davis, S., Sato, T. N., and Yancopoulos, G. D., Requisite role of angiopoietin-1, a ligand for the TIE2 receptor, during embryonic angiogenesis, Cell, 87, 1171-1180 (1996).

40. Maisonpierre, P. C., et al., Angiopoietin-2, a natural antagonist for Tie02 that disrupts in vivo angiogenesis. Science, 277, 55-60 (1997).

41. Wang, H. U. et al. (1998) cell 93:741.

42. Kitsukawa, T. et al. (1995) Development 121:4309.

43. Sierra-Honigmann, M. R., et al. (1998) Science 281: 1683.

44. Tachibana, K., et al. (1998) 393:591.

45. Cao, Y., Ji, R. W., Davidson, D., Schaller, J., Marti, D., Sohndel, S., McCance, S. G., O'Reilly, M. S., Llinas, M., and Folkman. J. (1996). J. Biol. Chem., 271, 29461-29467.

46. O'Reilly, M. S., Boehm, T., Shing, Y. et al. Cell, 88, 277-285, 1997.

47. Hynes, R. O. 1992. Integrins: versatility, modulation, and signalling in cell adhesion. Cell, 69:11-25.

48. Pytela, R., Pierschbacher, M. D., and Ruoslahti, E., 1985, A 125/115 kDa cell surface receptor specific for vitronectin interacts with the arginine-glycine-aspartic acid adhesion sequence derived from fibronectin. Proc. Natl. Acad. Sci. USA 82: 5766-5770.

49. Kireeva, M. L., Lam, S. C -T., and Lau, L. F. 1998, Adhesion of Human umbilical vein endothelial cells to the immediate-early gene product Cyr61 is mediated through integrin αvβ33. J. Biol. Chem. 273: 3090-3096.

50. Friedlander, M., Brooks, P. C., Shaeffer, R. W>, Kincaid, C. M., Varner, J. A., and Cheresh, D. A. 1995. Definition of two angiogenic pathways by distinct αβ integrins. Science 270: 1500-1502.

51. Brooks, P. C., Montgomery, A. M., Rosenfeld, M., Reisfeld, R. A. Hu, T., Kier, G., at al. 1994. Integrin αvβ3 antagonists promote tumour regression by inducing apoptosis of angiogenic blood vessels. Cell 79: 1157-1164.

52. Piali, L., Hammel, P., et al. 1995. CD31/PECAM-1 is a ligand for alpha v beta 3 integrin involved in adhesion of leukocytes to endothelium. J Cell. Biol., 130: 451-60.

53. Koivunen, E., Wang, B., and Tuoslahti, E., (1995) Phage Libraries Displaying Cyclic peptides with different ring sizes: Ligand specificities of the RGD-directed integrins. Bio/Technology Vol. 13, 1995,265-270.

54. Pierschbacher, M. D., and Rhuoslahti, E., Influence of stereochemistry of the sequence Arg-Gly-Asp-Xaa on binding specificity in cell adhesion. J. Biol. Chem. 262: 17294-17298, 1987

55. Tselepsis, V H, Green, L J, Humphries, M. J. An RGD to LDV motif conversion within the disintegrin kistrin generates an integrin antagonist that retains potency but exhibits altered receptor specificity. Evidence for a functional equivalence of acidic integrin-binding motifs. 1997 J. Biol. Chem. 272: 21341-21348.

56. Arap, W., Pasqualini, R., and Ruoslahti, E., Cancer Treatment by targeted drug delivery to tumour vasculature in a mouse model. 1998, Science, 279: 377-380.

57. Cooper, R. A., Wilks, D. P., Logue, J. P., Davidson, S. E., Hunter, R. D., Roberts, S. A., and West, C. M. High tumour angiogenesis is associated with poorer survival in carcinoma of the cervix treated with radiotherapy. Clin. Cancer res. 1998 $(11): 2795-800.

58. Brattstrom, D., Bergqvist, M, Larsson A, Holmertz, J., Hesselius P., Rosenberg L., Brodin, O., and Wagenuis G., Basic fibroblast growth factor and vascular endothelial factor in sera of non-small cell lung cancer patients. Anticancer Res. 1998 18(2A): 1123-7.

59. Landriscina, M., Cassano, A., Ratto, C., Longo, R., Ippoliti, M., Palazzotti, B., Crucitti, F., and Barone, C., Quantitative analysis of basic growth factor and vascular endothelial growth factor in human colorectal cancer, Br. J. Cancer, 1998, 78(6):765-70.

60. Eppenberger, U., Kueng, W., Schlaeppi, J M, Roesel, J L, Benz, C., Mueller, H., Matter A., Zuber, M., Luescher K., Litschgi, M, Schmitt, M, Koekens, J A, Eppenberger-Castori, S, Markers of tumour angiogenesis and proteolysis independently define high- and low-risk subsets of node-negative breast cancer patients. J. Clin. Oncol. 1998 16(9):3129-36.

61. Ogura, Y., Sato, K., Kato, T., Sato, K., and Enomoto, K., Immunohistochemical analysis of expression of angiogenic factors and tumour angiogenesis in superficial bladder cancer. Nippon Hinyokika Gakkai Zasshi 1998 89(5):529-37.

62. De Jonfg, J S, van Diest, P J, van der Valk P, Baak J P, Expression of growth factors, growth inhibiting factors and their receptors in invasive breast cancer. II:Correlations with proliferation and angiogenesis. J Pathol. 1998 184(1):53-57.

63. Takanami, I., Tanaka, F., Hashizune, T. and Kodaira, S., Vascular endothelial growth factor and its receptor correlate with angiogenesis and survival in pulmonary adenocarcinoma. Anticancer Res. 1997, 17:2811-2814.

64. Takahashi, Y., Cleary, K R, Mai, M, Kitadai, Y., Bucana, C D, Ellis, L M. Significance in cessel count and vascular endothelial growth factor and its receptor (KDR) and intestinal-type gastric cancer. Clin. Cancer Res. 1996 2(10):1679-1684.

65. Gasparini, G., Brooks, P C, Biganzoli, E., Vermeulen, P B, Bonoldi, E., Dirix, L Y, Ranieri G., Miceli, R., and Cheresh, D A. Vascular integrin alpha(v)beta3: a new prognostic indicator in breast cancer. Clin. Cancer Res. 1998 4(11): 2625-34.

66. Zhu, Z., Rockwell, P., Lu D., Kotanides. H., Pytowski, B., Hicklin, D J, Bohlen, P., and Witte, L, inhibition of vascular endothelial growth-factor induced receptor activation with anti-kinase insert fdomain-containing receptor single-chain antibodies from a phage display library. Cancer Res, 1998 58(15): 3209-3214; U.S. Pat. No. 5,747,651.

67. Witte, L., Hicklin, D J, Zhu, Z., Pytowski, B., Kotanides, H., Rockwell, P., and Bohlen, P. Monoclonal antibodies targeting the VEGF receptor-2 (Flk-1/KDR) as an anti-angiogenic therapeutic strategy. Cancer and Metastasis Reviews, 1998 17: 155-161.

68. Asano, M., Yukita, A., Matsumoto, T., Kondo, S., and Suzuki, H. Inhibition of tumour growth and metastasis by an immunoneutralizing monoclonal antibody to human vascular endothelial growth factor/vascular permeability factor 121. Cancer Res., 55:5296-5301 (1995).

69. Genentech

70. Techniclone, U.S. Pat. No. 5,776,427, and U.S. Pat. No. 5,660,827

71. Roeckl, W., Hecht, D., Sztajer, H., Waltenberger, J., Yayon, A., Weich, H A. Differential binding characteristics and cellular inhibition by soluble VEGF receptors 1 and 2. Exp. Cell. Res. 1998 241(1):161-170.

72. Lin, P., Sankar, S., Shan, S., Dewhirst, M W, Polverini, P J, Quinn, T Q, Peters K G, Inhibition of tumour growth by targeting tumour endothelium using a soluble vascular endothelial growth factor receptor. Cell Growth Differ. 1998 9(1):49-58.

73. WO-09816551,

74. Repligen

75. Sun, L., Tran, N., Tang, F, App, H., Hirth, P., McMahon, G., Tang, C., Synthesis and biological evaluations of 3-sustitutedindolin-2-ones: a novel class of tyrosine kinase inhibitors that exhibit selectivity toward particular tyrosine kinases. J. Med. Chem. 1998, 41(14): 2588-2603.; WO-09807695; U.S. Pat. No. 5,792,783.

76. The Burnam Institute, licensed to Integra, U.S. Pat. No. 5,695,997;

77. Scripps Research Institute. WO-09745137; WO-09745447

78- La Jolla Cancer Research Center, 5759996, U.S. Pat. No. 5,695,997

79. Maerck KgaA; WO-09827112.

80. The Burnam Institute, licensed to Integra; U.S. Pat. No. 5,827,821.

81. Carron, C P, Meyer, D M, Pegg, J A, Engleman, V W, Nickols, M A, Settle, S L, Westlin, W F, Ruminski, P G, Nickols, G A, A peptidomimetic antagonist of the integrin alpha(v)beta3 inhibits Leydig cell tumour growth and the development of hypercalcemia of malignancy. Cancer Res. 1998 58(9): 1930-1935.

82. Merck & Co Inc: WO-09831359; WO-09818461; WO-09708316.

83. Jadhav, P K, Eyemann, C J, Smallheer, J M, Woerner F J, Danaker, W F, Buynitzky, J S, Harlow, P P, Corjay, M H, Wexler, R R, Mousa, S A: Design and synthesis of spiroisoxazoline based alpha-V-beta-3 antagonists. Int Symp. Med. Chem. 1998 15^{th} Edinburgh 93.

84. Ixsys, WO-09833919.

85. Gutheil, J C, Campbell, T N, et al. Phase I study of Vitaxin, an anti-angiogenic humanized monoclonal antibody to vascular integrin alpha(v)beta3. Proc Am Soc Clin Oncol. 1998 34 May 16-19 Abs 832.

86. Sipkins, D A, Cheresh, D A, Kazemi, M R, Nevin, L M, Bednarski, M D, Li, K C: Detection of tumor angiogenesis in vivo by alphavbeta3-targeted magnetic resonance imaging. Nat Med 1998 4 5 623-626.

87. Antisoma: U.S. Pat. No. 5,395,609.

88. Sivolapenko, G B, Skarlos, D., Pectasides, D., Stathopoulou, E., Milonakis. A., Sirmais, G., Stuttle, A., Courtenay-Luck, N S, Konstantinides, K., and Epenetos, A A. Imaging of metastatic melanoma utilising a technetium-99m labelled RGD-containing synthetic peptide. Eur J Nuci Med, 25, 1998, 1383-1389.

89. Seventh annual IBC international conference on antibody engineering: New technology, application & commercialization. Coronado, Dec. 2-4, 1996 Iddb Meeting Report 1996

90. Antisoma: U.S. Pat. No. 5,628,979

91. Rouschias, G. Chem. Rev. 1974, 74, 531.

92. Fergusson, J. E. Coord. Chem. Rev. 1966, 1, 459.

93. J Med Chem. 1996. 39. 1372-1382

94. Gitay-Goren, H. et al. J. Biol. Chem. 267 (9): 6093-6098, 1992.

95. Landquist, J. K. (1956) J. Chem. Soc. 1956, 4237-4245.

96. Sin, N., Meng, L., Wang, M. Q. W., Wen, J. J., Bornmann, W. G., and Crews, C. M. (1997). The antiangiogenic agent fumagillin covalently binds and inhibits the methionine aminopeptidase. MetAP-2. Proc. Natl. Acad. Sci. USA 94, 6099-6103.

97. Ingber, D, et al. Nature 348, 555 (1990).

98. Ramakrishnan, S., Olson, T. A., Bautch, V. L., and Mohanraj, D., Cancer res. 1324-1330, 1996.

99. Soker, S., Gollamudi-Payne, S., Fidder, H., Charmahelli, H., and Klagsbni, M., J. Biol. Chem., 272, 1997, 31582-31588.

100. Eckelman W C, Meinken G, Richards P. 99mTc-human serum albumin. J Nuci Med 12, 1971, 707-710

101. Rhodes B A, Burchiel S W. Radiolabeling of antibodies with technetium-99m in radioimmunoimaging and radioimmunotherapy, Rhodes B A and Burchiel S W eds, Elsevier

102. Fritzberg A R. Advances in 99mTc-labeling of antibodies. Nucl Med Stuttgart, 26, 1987, 7-1

103. Kitsukawa, T. et al. (1995) Development 121:4309.

104. Antisoma: U.S. Pat. No. 5,628,979

105. J Med Chem. 1996, 39, 1372-1382

106. Gitay-Goren, H. et al. J. Biol. Chem. 267 (9): 6093-6098, 1992.

107. Ingber, D, et al. Nature 348, 555 (1990).

We claim:

1. A compound for the imaging and treatment of angiogenesis of the following formula (I):
A-(B)n-C (I)
wherein
A is a chelator moiety capable of complexing a radionuclide metal or a moiety capable of binding to a halogen;
B is a spacer group;
C is an angiogenesis targeting molecule; and
n is selected from the integers 0 and 1.

2. A compound according to claim 1, wherein C is fumagillin or an analogue thereof capable of localizing at sites of angiogenesis.

3. A compound according to claim 1, wherein C is a peptide that targets KDR/Flk-1 receptor, as an agonist or antagonist.

4. A compound according to claim 3 wherein C:
incorporates the peptidic sequence R-X-K-X-H, or R-X-K-X-H and I-X-X-I through a linker and flanked by amino acids, amino acid derivatives, or other molecular derivatives on each side, in a straight chain, branched, or cyclized fashion existing in the free acid, amide or capped or salt form; or
includes a group that acts as a peptidomimetic to said peptidic sequences.

5. A compound according to claim 1, wherein C:
is a peptide that targets Flk-1 receptor, the peptide incorporating the peptidic sequence D-E-X-X-E in a straight chain, branched, or cyclized fashion, the peptide existing in the free acid, amide capped or salt form; or
includes a group that acts as a peptidomimetic to said peptidic sequence.

6. A compound according to claim 1, wherein C is a peptide that targets Tie-1 or Tie-2 receptor.

7. A compound according to claim 1 wherein C targets a molecule selected from the group comprising: integrin receptors α1β1, α2β1, α4β1, α5β1, ephrin receptor eph B4, laminin A receptors, VEGF₁₆₅ receptor neurophilin, leptin receptor OB-Rβ and the chemokine receptor CXCR-4.

8. A compound according to claim 1 wherein C targets integrin receptors αvβx (x=3 or 5).

9. A compound according to claim 1 wherein C is selected from the group comprising osteopoietin, PECAM-1, fibronectin, vitronectin, Cyr-61, or pronectin-V.

10. A compound according to claim 1 wherein C is selected from the group comprising angiopoietin-1, angiopoietin-2, MMPs, angiostatin, endostatin and ephrin-B2.

11. A compound according to claim 1 wherein C is selected from the group comprising peptides, molecules and peptidomimetic derived from angiostatin, endostatin, angiopoietin-1, angiopoietin-2 and ephrin-B2.

12. A compound according to claim 1 wherein C includes the sequence R-G-D-S existing in the free acid, amide capped or salt form.

13. A compound according to claim 1 wherein C includes the sequence R-G-D-S bonded to other amino acids, amino acid derivatives, or molecules arranged in a straight chain, branched or cyclized manner, existing in the free acid, amide capped or salt form.

14. A compound according to claim 1 wherein C includes the sequence dR-G-dD-dS bonded to other amino acids, amino acid derivatives or molecules arranged in a straight chain, branched or cyclized manner, existing in the free acid, amide capped or salt form.

15. A compound according to claim 1 wherein C includes the sequence L-D-V bonded to other amino acids, amino acid derivatives or molecules arranged in a straight chain, branched or cyclized manner, existing in the free acid, amide capped or salt form.

16. A compound according to claim 1 wherein C includes the sequence NGR or RPK bonded to other amino acids, amino acid derivatives or molecules arranged in a straight chain, branched or cyclized manner, existing in the free acid, amide capped or salt form.

17. A compound comprising a metal chelating moiety and a moiety that binds to sites of angiogenesis.

18. A compound according to claim 17 including the sequence dmG-B-C(acm)-D-X-X, where B=dimethyl-p-amidinophenylalanine, dimethylarginine, or dimethyllysine.

19. A compound according to claim 17 including the sequence dmG-B-C(acm)-Z, where B=dimethyl-p-amidinophenylalanine, dimethylarginine, or dimethyllysine, and Z=homocysteine or 4-aminophenylacetic acid.

20. A compound according to claim 17 including the sequence dmB-G-C(acm)-D-X-X where B=dimethyl-p-amidinophenylalanine, dimethylarginine, or dimethyllysine.

21. A compound according to claim 17 including the sequence dmB-G-C(acm)-Z, where B=dimethyl-p-amidinophenylalanine, dimethylarginine, or dimethyllysine, and Z=homocysteine or 4-aminophenylacetic acid.

22. A compound for the imaging and treatment of angiogenesis comprising VEGF labeled with an isotope of iodine, technetium, rhenium or an active ester of a metal chelate.

23. A compound for the imaging and treatment of angiogenesis comprising angiostatin, endostatin, angiopoietin-1, angiopoietin-2, PECAM-1, MMPs, ephrin-B2, osteopoietin, fibronectin, vitronectin, Cyr-61 or pronectin-V labeled with an isotope of iodine, technetium or rhenium.

24. A compound according to claim 17, wherein A is a chelator of the formula: 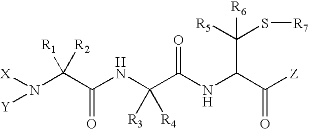
wherein
X is a linear or branched, saturated or unsaturated C₁₋₄alkyl chain that is optionally interrupted by one or two heteroatoms selected from N, O, and S, and is optionally substituted by one or more substituents selected from halogen, hydroxyl, amino, carboxyl, C₁₋₄alkyl, aryl, and C(O)Z;
Y is H or a substituent defined by X;
X and Y may together form a 5- to 8-membered saturated or unsaturated heterocyclic ring optionally substituted by one or more substituents selected form halogen, hydroxyl, amino, carboxyl, oxo, C₁₋₄alkyl, aryl, and C(O)Z;
R¹ and R⁴ are selected independently from H, carboxyl, C₁₋₄alkyl, C₁₋₄alkyl substituted with a substituent selected from hydroxyl, amino, sulfhydryl, halogen, carboxyl, C₁₋₄alkoxycarbonyl, and aminocarbonyl, an alpha carbon side chain of a D- or L-amino acid other than proline, and C(O)Z;
R⁵ and R⁶ are selected independently from H, carboxyl, amino, C₁₋₄alkyl, C₁₋₄ alkyl substituted by a substituent selected from hydroxyl, carboxyl, amino, and C(O)Z;
R⁷ is selected from H and a sulfur protecting group; and
Z is selected from a hydroxyl, alkoxy, and an amino acid analogue.

25. A compound according to claim 1 or 2, wherein n is 0.

26. A compound according to claim 1 or 2, wherein n is 1.

27. A compound according to any one of claims 1-17, in a form complexed with a diagnostically or therapeutically useful radionuclide metal.

28. A compound according to claim 22, wherein the radionuclide metal is selected from the group comprising ^{99m}Tc, ⁹⁹Tc ⁶⁴Cu, ⁶⁷Cu, ⁹⁷Ru, ¹⁰⁹Pd, ¹⁸⁶Re, ¹⁸⁸Re, ¹¹¹In, ^{113m}In, ¹⁵³Gd, 90Y, ¹⁵³Sm, 166Ho, ¹⁹⁸Au, ¹⁹⁹Au, ⁹⁰Sr, ⁸⁹Sr, 105Rh, ²⁰¹Tl, ⁵¹Cr, ⁶⁷Ga, ⁵⁷Co, ⁶⁰Co.

29. A compound according to any one of claims 1-17, in a form complexed with a halogen.

30. A compound according to claim 23 wherein the halogen is selected from the group comprising ¹²³I, ¹²⁵I, ¹³¹I and ¹⁸F.

31. A method of imaging an angiogenic site in a animal comprising the step of administering a diagnostically effective amount of a composition comprising a compound according to one of claims 1-30.

32. A method of treating cancer in a patient comprising the step of administering a therapeutically effective amount of a composition comprising a compound according to claims 1-30.

33. A method of staging a tumour in a patient comprising the step of administering a therapeutically effective amount of a composition comprising a compound according to claims 1-30.

34. A composition of matter comprising the compound of claim 1 and a stannous ion.

35. A kit for preparing a radiopharmaceutical preparation, said kit comprising a sealed vial containing a predetermined quantity of a peptide or molecular reagent according to claim 1 and a sufficient amount of reducing agent to label said reagent with Tc-99m.
